(19) United States
(12) Patent Application Publication
(10) Pub. No.: US 2024/0356370 A1
(43) Pub. Date: Oct. 24, 2024

(54) METHOD AND DEVICE FOR ENERGY TRANSFER

(71) Applicant: National University of Singapore, Singapore (SG)

(72) Inventors: Jerald Yoo, Singapore (SG); Yilong Dong, Singapore (SG); Jiamin Li, Singapore (SG)

(21) Appl. No.: 18/686,844

(22) PCT Filed: Aug. 26, 2022
(86) PCT No.: PCT/SG2022/050610

§ 371 Date: Feb. 26, 2024

(30) Foreign Application Priority Data
Aug. 27, 2021 (SG) 10202109404Q

(51) Int. Cl. H02J 50/05; H02J 50/00; H02J 50/40

(52) U.S. Cl. CPC H02J 50/05; H02J 50/001; H02J 50/40

(57) ABSTRACT

A power transmitter 201 is disclosed. In an example embodiment, the power transmitter comprises: a signal generator operable to generate an electrical signal comprising an adjustable frequency and duty cycle; one or more first transmitter electrodes 209 arranged to be electrically coupled to a living being body 105 for transmission of the electrical signal via the living being body 105 to one or more power receiver(s) 301a; one or more second transmitter electrode(s) 211 arranged to be electrically coupled to the living being body 105, and operable to receive an indication of power received by the at least one power receiver 301a via the living being body; and a controller 203 configured to control the signal generator to adjust the frequency and duty cycle of the electrical signal in response to the indication of power received. A power receiver 301a and methods of power transmission and power receiving are disclosed.

### FIELD AND BACKGROUND

This disclosure invention relates to a method and device for energy transfer, more particularly but not exclusively, for powering wearable or implantable electronics using energy transmission via the human body.

Body area networks require the efficient powering of diverse wearable nodes, such as earbuds, smart band-aids, and electrocardiography (ECG) sensors to name but a few.

Some wearable nodes are provided with a battery (e.g. hubs) but usually they have no storage capability under mm-scale. This mandates remote powering from, for example, a smartphone or a smart watch, providing power to rest of the nodes around the body upon request. When it comes to powering in the body area, conventional powering methods as schematically illustrated in FIG. 1 have shortcomings. Specifically, Photovoltaics 11 require light exposure, which is difficult on wearables covered with clothing; piezo/triboelectric harvesters 13 require movement, which is not always guaranteed; and RF-based power transfer 15 is often hindered by the body shadowing effect, particularly under non-line of sight situations.

Recently, Body Coupled Powering (BCP) using the human body itself as the medium has been shown to enable power delivery to wearable electronics.

It is desirable to provide a method and device for powering wearable nodes which addresses at least one of the drawbacks of the prior art and/or to provide the public with a useful choice.

### SUMMARY

In a first aspect, there is provided a power transmitter. The power transmitter comprises: a signal generator operable to generate an electrical signal comprising an adjustable frequency and duty cycle; a first transmitter electrode arranged to be electrically coupled to a living being body for transmission of the electrical signal via the living being body to at least one power receiver; a second transmitter electrode arranged to be electrically coupled to the living being body, and operable to receive an indication of power received by the at least one power receiver via the living being body; and a controller configured to control the signal generator to adjust the frequency and duty cycle of the electrical signal in response to the indication of power received. By adjusting the frequency and duty cycle of the electrical signal according an indication of power received by the at least one power receiver, the frequency and power transmitted can be optimized for the position and requirements of the receiver.

Preferably, the electrical signal comprises a plurality of components, and the one or more first electrodes are arranged to be electrically coupled to the living being body for transmission of each of the plurality of components of the electrical signal via the living being body to a corresponding one of a plurality of power receivers. Further, the one or more second electrodes are operable to receive an indication of power received by each of the plurality of power receivers via the living being body, and the controller is configured to control the signal generator to adjust a component frequency and a component duty cycle of each of the plurality of components of the electrical signal in response to the indication of power received by the corresponding power receiver.

In a specific embodiment, the signal generator further comprises a power generating circuit including: a voltage multiplier circuit arranged to receive an input supply voltage, the voltage multiplier circuit comprising a plurality of cascaded voltage multipliers arranged to directly generate corresponding multiplier output signals; and a driving circuit configured to generate the electrical signal with a specific output voltage swing in response to the corresponding multiplier output signals, the specific output voltage swing being greater than the input supply voltage. Preferably, the corresponding multiplier output signals include a driver supply voltage, at least one intermediate biasing signals, and a plurality of clock signals for the driving circuit. This provides the required drivability for a high voltage (HV) driving circuit.

In a second aspect, there is provided an energy transfer apparatus. The energy transfer apparatus comprises a power transmitter comprising a signal generator operable to generate an electrical signal comprising an adjustable frequency and duty cycle; a first transmitter electrode arranged to be electrically coupled to a living being body for transmission of the electrical signal via the living being body to at least one power receiver; a second transmitter electrode arranged to be electrically coupled to the living being body, and operable to receive an indication of power received by the at least one power receiver via the living being body; and a controller configured to control the signal generator to adjust the frequency and duty cycle of the electrical signal in response to the indication of power received. The energy transfer apparatus further comprises the at least one power receiver. The at least one power receiver comprises: one or more power receiver electrodes arranged to be electrically coupled to the body of the living being body, the one or more power receiver electrodes operable to receive the transmitted electrical signal via the body; at least one rectifier for rectifying the electrical signal into at least one rectified electrical signal; a DC-DC converter operable to convert the at least one rectified electrical signal to generate a recovered power signal, wherein the DC-DC converter is operable to determine the power received by the power receiver; and one or more data transmitter electrodes arranged to be electrically coupled to the living being body for transmission of an indication of the power received by the power receiver to the power transmitter.

In a specific embodiment, the one or more power receiver electrodes are further operable to receive a body-coupled ambient energy electrical signal via the body.

Preferably, the power receiver further includes a first rectifier for rectifying the transmitted electrical signal into a first rectified electrical signal; a second rectifier for rectifying the body-coupled ambient energy electrical signal into a second rectified electrical signal, and wherein the DC-DC converter is operable to convert the first rectified electrical signal and the second electrical signal concurrently to generate the recovered power signal.

Advantageously, the power receiver further comprises a controller, wherein the controller is arranged to adjust an inductor charging time of the DC-DC converter to enable an input power of the DC-DC converter to reach a maximum value. It is also envisaged that DC-DC converter includes a power evaluation circuit and is operable between a load regulation mode for delivering regulated power to a load and a power evaluation mode for determining the power received by the power receiver. Preferably, the DC-DC converter is operable between the load regulation mode and the power evaluation mode responsive to a voltage level of the recovered power signal. In a specific embodiment, the power received by the power receiver is determined as a function of an inductor discharge time of the DC-DC converter. It is also envisaged that the power received by the power receiver may be determined as an average received power.

In a third aspect, there is provided a power receiver. The power receiver comprises: one or more receiver electrodes arranged to be electrically coupled to a body of a living being, the one or more receiver electrodes operable to receive an electrical signal via the body, the electrical signal comprising both transmitted components and harvested ambient energy components; a first rectifier for rectifying the transmitted components into a first rectified electrical signal; a second rectifier for rectifying the harvested ambient energy components into a second rectified electrical signal; and a DC-DC converter operable to convert the first rectified electrical signal and the second rectified electrical signal concurrently to generate a combined recovered power signal. By concurrently converting rectified signals corresponding to transmitted power and harvest energy, power efficiency of the power transmission may be optimised by minimising the transmitted power required.

In a specific embodiment, the DC-DC converter further comprises a power evaluation circuit and is further operable to determine a power received by the power receiver.

Preferably, the power receiver further comprises one or more further receiver electrodes arranged to be electrically coupled to the living being body for transmission of an indication of the power received by the power receiver to a power transmitter.

In a fourth aspect, there is provided a method of transmitting electrical power via one or more first transmitter electrodes coupled to a body of a living being. The method comprises generating, by a signal generator having an adjustable frequency and duty cycle, an electrical signal; receiving an indication of power received by at least one power receiver via one or more second transmitter electrodes coupled to the living being body; and adjusting the frequency and duty cycle of the electrical signal in response to the indication of power received.

In a specific embodiment, the method further comprises generating the electrical signal with a plurality of components, each component operable to transmit electrical power to a different power receiver of a plurality of power receivers; receiving an indication of power received by each of the plurality of power receivers via the one or more second transmitter electrodes; adjusting a component frequency and a component duty cycle of each of the plurality of components of the electrical signal in response to the indication of power received by the corresponding power receiver. Preferably, the method comprises time-division multiplexing the plurality of components to generate the electrical signal.

Advantageously, the method further comprises receiving an input supply voltage at a voltage multiplier circuit, the voltage multiplier circuit comprising a plurality of cascaded voltage multipliers; directly generating, by the plurality of cascaded voltage multipliers, corresponding multiplier output signals; generating the electrical signal with a specific output voltage swing in response to the corresponding multiplier output signals, the specific output voltage swing being greater than the input supply voltage. It is envisaged that the multiplier output signals may include a driver supply voltage, at least one intermediate biasing signal, and a plurality of clock signals for a driving circuit.

In a fifth aspect, there is provided a method of transferring energy via a body of a living being, the method comprising: transmitting electrical power via one or more first transmitter electrodes coupled to a body of a living being, including by generating, using a signal generator having an adjustable frequency and duty cycle, an electrical signal, receiving an indication of power received by at least one power receiver via one or more second transmitter electrodes coupled to the living being body, and adjusting the frequency and duty cycle of the electrical signal in response to the indication of power received. The method further comprises receiving, at a power receiver, the transmitted electrical signal via one or more power receiver electrodes coupled to the living being body; rectifying the electrical signal into at least one rectified electrical signal;

converting the at least one at least one rectified electrical signal to generate a recovered power signal; determining a power received by the power receiver; and transmitting an indication of the power received to the power transmitter via one or more data transmitter electrodes coupled to the living being body.

In a specific embodiment, the method further comprises converting, at a DC-DC converter, the at least one rectified electrical signal to generate the recovered power signal, and adjusting an inductor charging time of the DC-DC converter to enable an input power of the DC-DC converter to reach a maximum value. Preferably the method further includes determining the power received by the power receiver at the DC-DC converter. It is envisaged that the power received by the power receiver may be determined as a function of an inductor discharge time of the DC-DC converter.

In a sixth aspect, there is provided a method of receiving power via one or more receiver electrodes coupled to a body of a living being. The method comprises receiving an electrical signal via the body, the electrical signal comprising both transmitted components and harvested ambient energy components; rectifying the transmitted components into a first rectified electrical signal; rectifying the harvested ambient energy components into a second rectified electrical signal; and converting the first rectified electrical signal and the second rectified electrical signal concurrently to generate a combined recovered power signal. Preferably, the method further includes determining a power received via the one or more receiver electrodes, and transmitting an indication of the power received via one or more further receiver electrodes coupled to the living being body.

In a seventh aspect, there is provided a power generation circuit comprising a voltage multiplier circuit arranged to receive an input supply voltage, the voltage multiplier circuit comprising a plurality of cascaded voltage multipliers arranged to directly generate corresponding multiplier output signals; and a driving circuit configured to generate a specific output voltage signal in response to the corresponding multiplier output signals, the specific output voltage signal having a greater voltage swing than the input supply voltage. In a specific embodiment, the corresponding multiplier output signals include a driver supply voltage, at least one intermediate biasing signal, and a plurality of clock signals for the driving circuit.

In an eighth aspect, there is provided a method of power generation. The method comprises receiving an input supply voltage; generating, by a plurality of cascaded voltage multipliers, corresponding multiplier output signals; generating, at a driving circuit, a specific output voltage signal in response to the corresponding multiplier output signals, the specific output voltage signal having a greater voltage swing than the input supply voltage. In a specific embodiment, the corresponding multiplier output signals include a driver supply voltage, at least one intermediate biasing signal, and a plurality of clock signals for the driving circuit.

In a ninth aspect, there is provided a transmitter comprising 1) an electrode 2) a power generator 3) feedback data recovery, which adjusts output power & frequency to a subset of receiving nodes based on the receiver feedback. The transmitter may further be capable of high voltage (HV) signal generation and the data recovery may include an amplifier, envelope detector, comparator and digital control.

In a tenth aspect, there is provided, a receiver comprising 1) an electrode 2) power recovery, 3) a data transmitter, which recovers, evaluates and feedbacks the recovered power level. The receiver may support frequency tuning. The receiver may further comprise a rectifier and be configured for multi-source power conversion. Further recovered power may be directed to secondary storage for evaluation and Feedback may be transmitted upon completion of evaluation.

In an eleventh aspect, there is provided a power transmitter which comprises:
a. a signal generator with adjustable frequency and duty cycle for power output and which is configured for the of high-voltage power output which eliminates level shifter(s), biasing circuit(s), and tapered buffer(s) which are needed otherwise, reducing power overhead;
b. an electrode(s) to couple the power output onto the human body;
c. an electrode(s) to couple the feedback signal from the human body to the data recovery module;
d. a data recovery module which amplifies and recovers the feedback data; and
e. a micro-controller which interprets the recovered data and adapts the output frequency and duty cycle of the signal generator.

In a twelfth aspect, there is provided a power receiver which comprises:
a. an electrode(s) to couple the received power from the human body to the rectifier module;
b. a rectifier which converts the transmitted power to DC power;
c. a rectifier which harvests EM signals coupled onto human body from the ambience; and
d. a DC-DC power converter which outputs a regulated voltage and performs power evaluation.

The power converter may extract power from both the body-coupled received power and the body-coupled harvested power concurrently, at the maximum power point of each source. The DC-DC power converter may integrate a secondary output path which evaluates the recovered power level. A data transmitter may be provided which encodes and transmits the information on the recovered power level. The data transmitter may only be enabled upon the completion of power evaluation, and may be self-disabled upon one transmission cycle

It should be appreciated that features relevant to one aspect may also be relevant to the other aspects.

### BRIEF DESCRIPTION OF THE DRAWINGS

Exemplary embodiments will now be described with reference to the accompanying drawings, in which:

FIG. 1 illustrates three conventional body powering methods;

FIG. 2 illustrates an energy transfer apparatus coupled to a user's body according to a preferred embodiment;

FIG. 3 illustrates the architecture of a power transmitter and first power receiver of the energy transfer apparatus of FIG. 2;

FIG. 4 illustrates a power output module of the power transmitter of FIG. 3;

FIG. 5 illustrates an all-in-one high voltage power generation circuit and its connection to and HV driver of the power output module of FIG. 4;

FIG. 6 illustrates a dual-source power converter and power evaluation and feedback controller of the first power receiver of FIG. 3;

FIG. 7a illustrates a pulse generation module of the dual-source power converter of FIG. 6;

FIG. 7b illustrates an inner loop enable signal generation circuit for the pulse generation module of FIG. 7a;

FIG. 8a schematically illustrates an exemplary portion of a module of the dual source power converter of FIG. 6 including control switch drivers and one adaptive level shifter (ALS);

FIG. 8b schematically illustrates at T1 delay module of the pulse generation module of FIG. 7a;

FIG. 8c schematically illustrates a T0 delay module of the pulse generation module of FIG. 7a;

FIG. 8d shows a method of determining T1 and T2 delays in the pulse generation module of FIG. 7a;

FIG. 8e shows a method of asynchronous maximum power point tracking (MPPT) performed to determine the T1 delay in the pulse generation module of FIG. 7a; FIG. 8f summarises the method of receiving power performed by the first power receiver of FIG. 3;

FIG. 9a illustrates the power evaluation and feedback control module of FIG. 6;

FIG. 9b illustrates the data transmitter of the power receiver of FIG. 3;

FIG. 9c illustrates the data receiver of the power transmitter of FIG. 3;

FIG. 10a illustrates an operation of power transfer between the power transmitter and the first power receiver of FIG. 3;

FIG. 10b summarizes the method of operation of FIG. 10a;

FIG. 11 illustrates an example of a total signal transmitted by the power transmitter of FIG. 3 including three different frequency components;

FIGS. 12a-c illustrate results for transmitter output power required (FIG. 12b), and body channel gain (FIG. 12c) for three receivers positioned at different positions on a body (shown in FIG. 12a);

FIG. 13 compares the fixed output of a transmitter with node-specific output according to the described embodiment;

FIG. 14 shows die micrographs of a prototype receiver and transmitter according to the described embodiment;

FIG. 15 illustrates measurement results of high voltage pulse generation at the prototype power transmitter of FIG. 14;

FIG. 16a illustrates the maximum power delivered using the prototype power transmitter of FIG. 14 when compared with a conventional 2.5 Vₚₚ output;

FIG. 16b illustrates power delivered with frequency adaptation compared with conventional fixed frequency power transmission;

FIG. 17 illustrates improved receiver power due to dual-source power recovery showing power recovered using dual-source recovery as well as power transmission recovery only with ⅓ TX output duty cycle; and

FIG. 18 illustrates results for a multinode test case where a TX (placed on arm,) delivers power to 3 RX (placed on forehead, hand and ear) of fixed power request ranging from 1 μW to 7 μW.

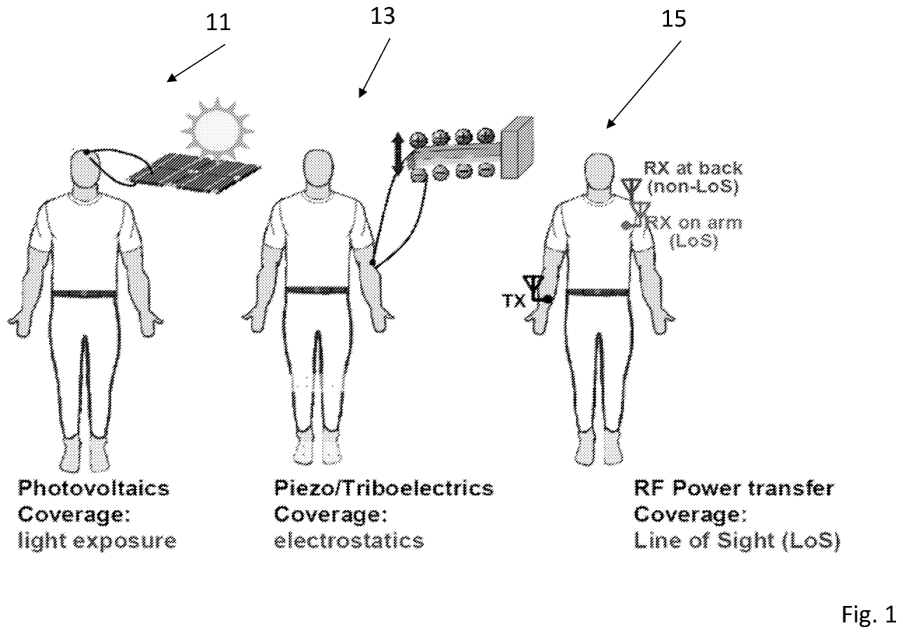

### DETAILED DESCRIPTION

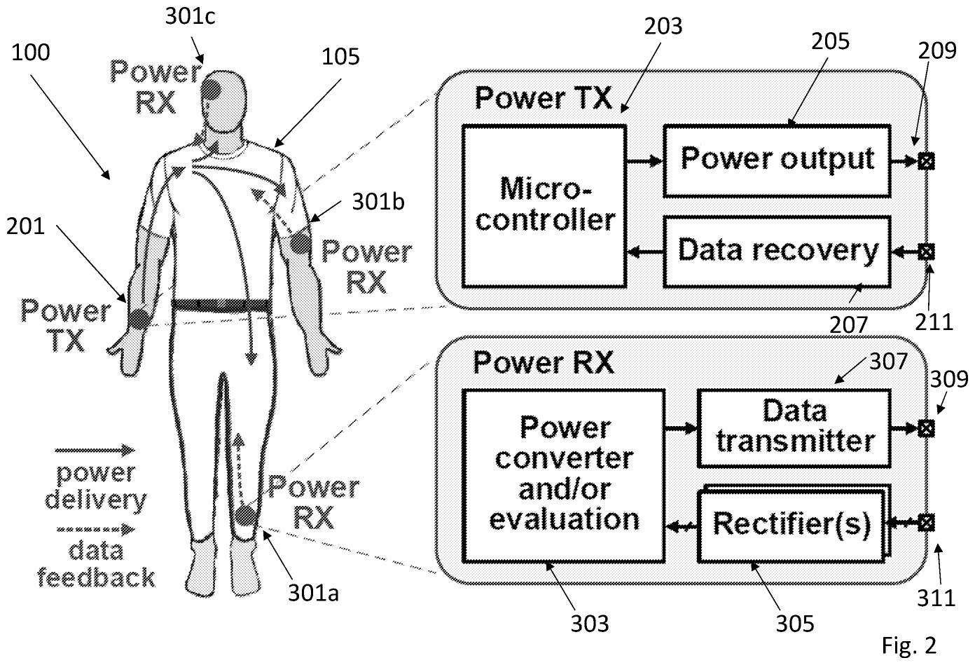

FIG. 2 illustrates an energy transfer apparatus 100 coupled to a user's body 105 according to a preferred embodiment for use in body coupled powering. The energy transfer apparatus 100 comprises a power transmitter 201 coupled to the user's body 105 and three power receivers 301a, 301b, 301c in the form of wearable or implantable electronic devices also coupled to different parts of the user's body 105. Specifically, the power transmitter 201 is coupled to a wrist of the right hand of the user's body 105, and a first power receiver 301a is attached to the left ankle, a second power receiver 301b is attached to the left arm, and a third power receiver 301c is attached to the head. As illustrated, the three power receivers 301a, 301b, 301c are operable to receive electrical power via an electrical signal transmitted by the power transmitter 201 via capacitive coupling via a body channel and to transmit data feedback to the power transmitter 201. As will become apparent from the below, in the described embodiment, the transmitter is configured to transmit specific frequencies to each of one of the receiver nodes using Time Division Multiplexing (TDM).

Independently from the power transmitter 201, the three power receivers 301a, 301b, 301c are also operable to harvest ambient EM waves from ambient electrical sources via electrical signals within the user's body 105, low-frequency ambient EM waves being observed to couple onto the human body, with the 50/60 Hz wave dominating. Both body coupled power transmission and harvesting of body coupled EM waves are discussed generally in J. Li, Y. Dong, J. H. Park, L. Lin, T. Tang and J. Yoo, “Body-Area Powering with Human Body-Coupled Power Transmission and Energy Harvesting ICs,” IEEE Transactions on Biomedical Circuits and Systems, doi: 10.1109/TBCAS.2020.3039191.

As illustrated in FIG. 2, the architecture of the power transmitter 201 includes a microcontroller (MCU) 203, a power output module 205 and a data receiver 207 configured to amplify and recover feedback data, the power output module 205 arranged to receive data from the MCU 203 and the data receiver 207 arranged to transmit data to the MCU 203. The power transmitter 201 further includes two electrodes 209, 211 via which the power transmitter 201 is coupled to the user's body 105, specifically a first electrode 209 which couples the power output module 205 to the user's body 105 and a second electrode 211 which couples the data receiver 207 to the user's body 105.

The architecture of the first power receiver 301a relevant to power receiving is also illustrated in FIG. 2 as an example (note that the load of the wearable device is not shown in FIG. 2). The first power receiver 301a includes a power converter and evaluation module 303, two rectifiers 305, and a data transmitter 307. The first power receiver 301a also includes two electrodes 309, 311 via which the first power receiver 301a is coupled to the user's body 105, specifically, a first electrode 309 which couples the data transmitter module 307 to the user's body 105 and a second electrode 311 which couples the two rectifiers 305 to the user's body 105.

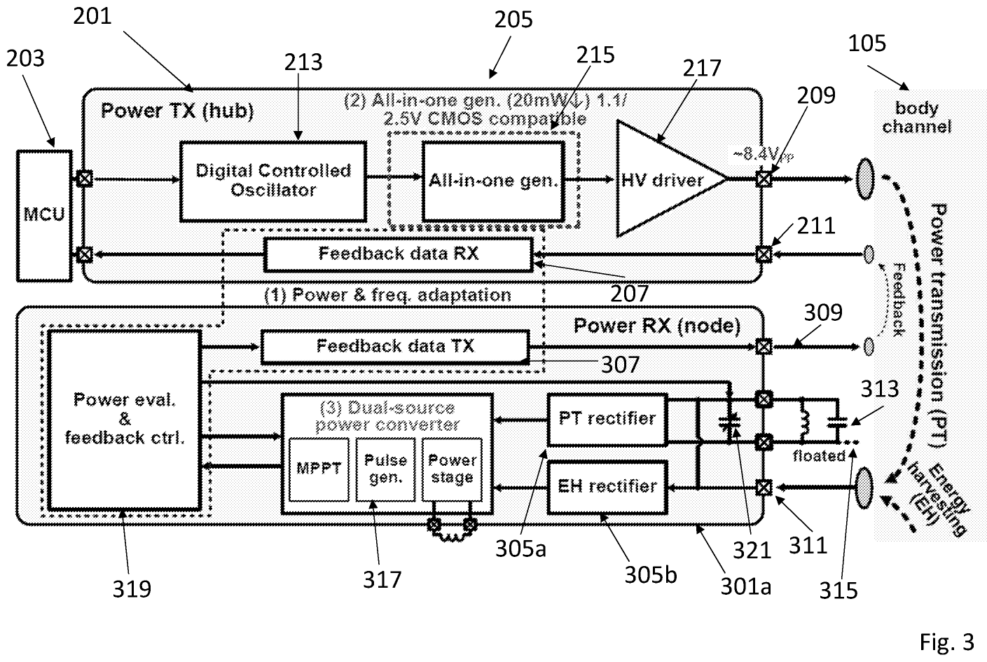

FIG. 3 illustrates the architecture of the power transmitter 201 and first power receiver 301a in more detail.

As will be appreciated from FIG. 3, the power output module 205 includes a signal generator in the form of a digitally controlled oscillator 213 which is operable to generate an electrical signal having an adjustable frequency and a duty cycle for power output—i.e. the amount of time at which the digitally controlled oscillator 213 outputs a particular frequency component of the electrical signal as a percentage of the total operation time of the digitally controlled oscillator 213—which is also adjustable. The power output module 205 also includes a high voltage power generation circuit 215 and a high voltage (HV) driver 217. The digitally controlled oscillator 213 is configured to receive control instructions from the MCU 203 and output the generated electrical signal to the high voltage power generation circuit 215 which, in turn outputs to the HV driver 217 and then to the user's body 105 via the electrode 209. The MCU 203 itself is configured to interpret recovered data and adapt the output frequency and duty-cycle of the digitally controlled oscillator 213 accordingly. It should be appreciated that the power transmitter 201 also includes a floating ground node which is not shown in FIG. 3.

Turning now to the first power receiver 301a, the first power receiver 301a includes an impedance boosting circuit in the form of a parallel L-C circuit 313 having a floating ground node 315 of the first power receiver 301a. The two rectifiers 305 include a power transmission rectifier 305a which is operable to convert power transmitted by the power transmitter 201 to DC power and an energy harvesting rectifier 305b which is operable to rectify ambient EM signals coupled to the user's body 105 in order to harvest them.

Suitable rectifiers suitable for use as the power transmission rectifier 305a and the energy harvesting rectifier 305b include those described in, for example, J. Li, Y. Dong, J. H. Park, L. Lin, T. Tang and J. Yoo, “Body-Area Powering with Human Body-Coupled Power Transmission and Energy Harvesting ICs,” IEEE Transactions on Biomedical Circuits and Systems, doi: 10.1109/TBCAS.2020.3039191 and H. Cho, J. Suh, H. Shin, Y. Jeon, C. Jung and M. Je, “An Area-Efficient Rectifier with Threshold Voltage Cancellation for Intra-Body Power Transfer,” 2019 IEEE International Symposium on Circuits and Systems (ISCAS), Sapporo, Japan, 2019, pp. 1-5, doi: 10.1109/ISCAS.2019.8702391. Alternatively, conventional cross-coupled rectifiers (see for example K. Kotani, A. Sasaki and T. Ito, “High-efficiency differential-drive CMOS rectifier for UHF RFIDs”, IEEE J. Solid-State Circuits, vol. 44, no. 11, pp. 3011-3018 November 2009.) or full-bridge rectifiers with appropriate transistor sizing may be employed rectify the corresponding signal. Design considerations guiding transistor sizing are explained in J. Li, Y. Dong, J. H. Park, and J. Yoo, “Body-Coupled Power Transmission and Energy Harvesting,” Nature Electronics, vol. 4, pp. 530-538, 2021. doi: 10.1038/s41928-021-00592-y.

Connections between the power transmission rectifier 305a and the electrode 311 as well as the power transmission rectifier 305a and the floating input node 315 are provided in the first power receiver 301a either side of the L-C circuit 313, the floating input node 315 forming an electrical signal return path from the first power receiver 301a to power transmitter 201 the via parasitic capacitance with the external ground for body-coupled power transmission. A connection between the energy harvesting rectifier 305b and the electrode 311 is also provided in the first power receiver 301 for harvesting energy from the user's body 105.

The power converter and/or evaluation module 303 includes a dual-source power converter 317, specifically a DC-DC power converter configured to output a regulated voltage and perform power evaluation. The power converter and/or evaluation module 303 receives inputs concurrently from both the power transmission rectifier 305a and the energy harvesting rectifier 305b and is configured to extract power from them at the maximum power point (MPPT) of each source. The power converter and evaluation module 303 also receives inputs from a power evaluation and feedback controller 319 also included in the first power receiver 301a.

The power evaluation and feedback controller 319 is in communication with the data transmitter 307. A connection is provided from the power evaluation and feedback controller 319 to control a variable capacitor 321 in the form of a capacitor bank which is connected in parallel with the parallel L-C circuit 313. The power evaluation and feedback controller 319 adjusts the variable capacitor 321 to tune the L-C circuit 313 to optimize power recovery and perform frequency rejection for the TDM as will be discussed in detail below.

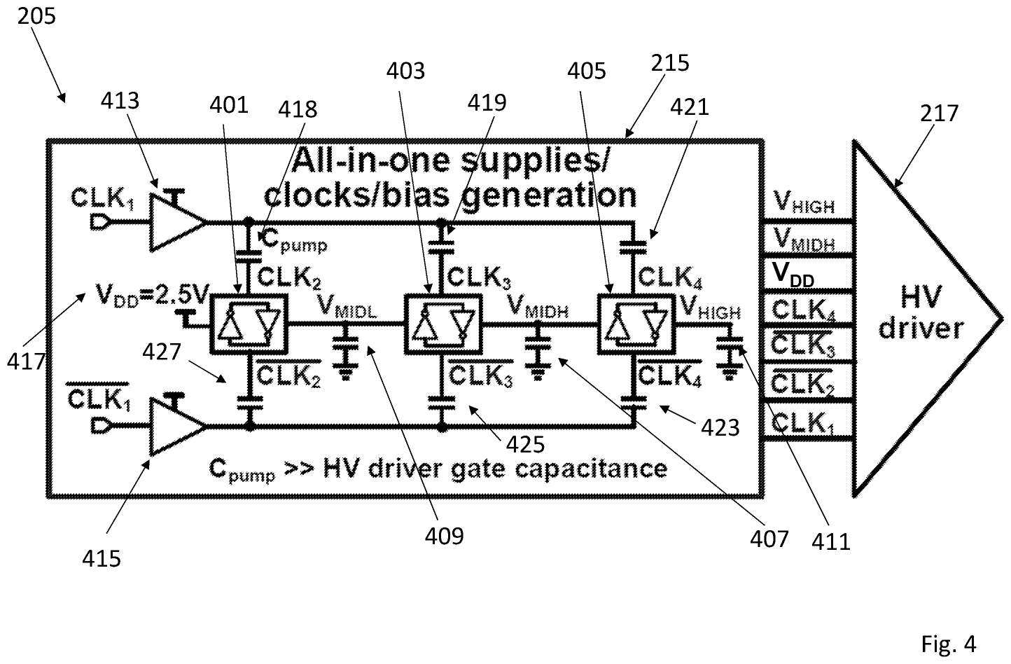

FIG. 4 illustrates the power output module 205 in more detail, in particular illustrating the arrangement of the high voltage power generation circuit 215.

The high voltage power generation circuit 215 includes three cascaded cross-coupled voltage multipliers 401, 403, 405, specifically a first voltage multiplier 401, a second voltage multiplier 403 and a third voltage multiplier 405. The input to the first voltage multiplier 401 is a supply voltage V_{DD} 417 which in the described embodiment is 2.5V. The output of the first voltage multiplier 401 is coupled to the input of the second voltage multiplier 403 and additionally to a first load capacitor 409. The output of the second voltage multiplier 403 is coupled to the input of the third voltage multiplier 405 and additionally to a second load capacitor 407, the output of the third voltage multiplier 407 is coupled to a third load capacitor 411.

The high voltage power generation circuit 215 further includes a first identity gate 413 taking a first clock signal CLK₁ as input and V_{DD} as a supply and a second identity gate 415 taking a first non-overlapping clock signal (which does not overlap with the first clock signal) C̅L̅K̅₁̅ as input and V_{DD} as a supply. Six capacitors 418, 419, 421, 423, 425, 427 are further included in the high voltage power generation circuit 215. Specifically, a first capacitor 418 couples the output of the first identity gate 413 to the first voltage multiplier 401 and provides a second clock signal CLK₂ to the first voltage multiplier 401, a second capacitor 419 couples the output of the first identity gate 413 to the second voltage multiplier 403 and provides a third clock signal CLK₃ to the second voltage multiplier 403, a third capacitor 421 couples the output of the first identity gate 413 to the third voltage multiplier 405 and provides a fourth clock signal CLK₄ to the third voltage multiplier 405, a fourth capacitor 427 couples the output of the second identity gate 415 to the first voltage multiplier 401 and provides a second non-overlapping clock signal C̅L̅K̅₂̅ to the first voltage multiplier 401, a fifth capacitor 425 couples the output of the second identity gate 415 to the second voltage multiplier 403 and provides a third non-overlapping clock signal C̅L̅K̅₃̅ to the second voltage multiplier 403, and a sixth capacitor 423 couples the output of the second identity gate 415 to the third voltage multiplier 405 and provides a fourth non-overlapping clock signal C̅L̅K̅₄̅ to the third voltage multiplier 405.

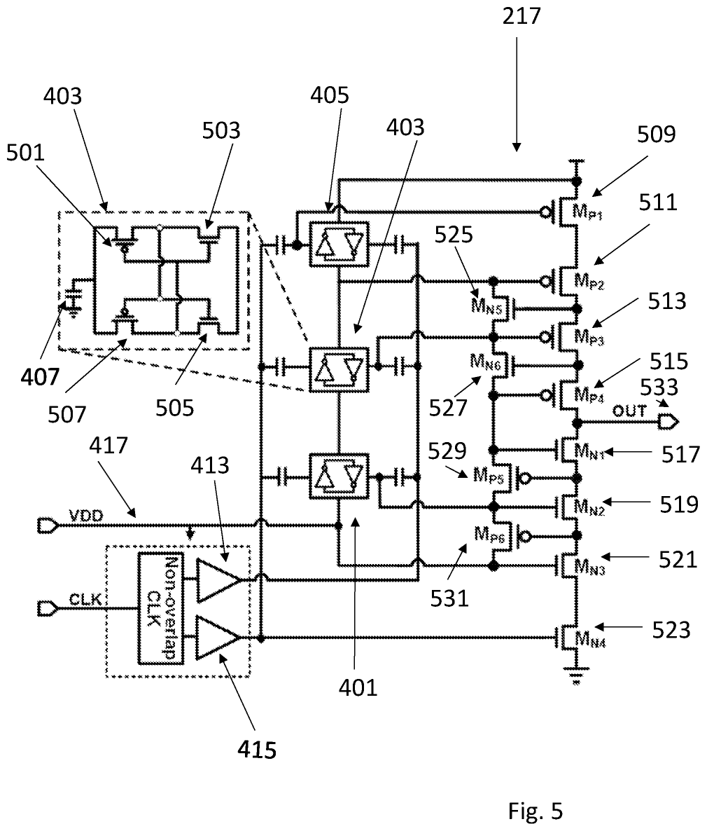

The structure of the second voltage multiplier 403 is illustrated in more detail in FIG. 5 as an example. The second voltage multiplier 403 comprises two coupled CMOS invertors, the output of each inverter comprising the input of the other.

Specifically, a first CMOS invertor includes a first PMOS transistor 501 connected to a first NMOS transistor 503 at their drain and gate terminals. A second CMOS invertor includes a second PMOS transistor 507 and a second NMOS transistor 505 connected at their drain and gate terminals. The first NMOS transistor 503 and the second NMOS transistor 505 are connected at their source terminals. The first PMOS transistor 501 and the second PMOS transistor 507 are also connected at their source terminals.

The gates of the first PMOS transistor 501 and the first NMOS transistor 503 are also coupled to the drain terminals of the second PMOS transistor 507 and the second NMOS transistor 505, while the gates of the second PMOS transistor 507 and the second NMOS transistor 505 are coupled to the drain terminals first PMOS transistor 501 and the first NMOS transistor 503.

The source terminals of the two PMOS transistors 501, 507 are coupled to the third voltage multiplier 405 and the second load capacitor 407 and, as such, provide an output signal from the second voltage multiplier 403 to the third voltage multiplier 405. The source terminals of the two NMOS transistors 503, 505 are coupled to the first voltage multiplier 401 and the first load capacitor 409 and, as such, receive an input signal to the second voltage multiplier 403. The drain terminals of the first PMOS transistor 501 and the first NMOS transistor 503 are further coupled to the output of the second identity gate 415 via the fifth capacitor 425 (via which the third non-overlapping clock signal C̅L̅K̅₃̅ is received). The drain terminals of the second PMOS transistor 507 and the second NMOS transistor 505 are coupled to the first identity gate 413 via the second capacitor 419 (via which the second clock signal CLK₂ is received). It should be appreciated that corresponding features are included in the first voltage multiplier 401 and the third voltage multiplier 405. These will not be explicitly described here for brevity.

As illustrated in FIG. 4, the high voltage power generation circuit 215, or multiplier circuit, outputs not only a supply voltage V_{HIGH} as an output signal from the third voltage multiplier 405 to the HV driver 217 but also output signals to the HV driver 217 corresponding to the second voltage multiplier 403 specifically, intermediate biasing signal V_{MIDH} output from the second voltage multiplier 403 as well as the output signals in the form of the first clock signal CLK₁, the second non-overlapping signal C̅L̅K̅₂̅, the third non-overlapping clock signal C̅L̅K̅₃̅ and the fourth clock signal CLK₄ as direct signals for driving the HV driver 217. In the described embodiment, these signals themselves provide the drivability required by the HV driver 217. In other words, the high voltage power generation circuit 215 enables buffers that are conventionally required to drive the next-stage transistors to be omitted.

FIG. 5 illustrates the all-in-one high voltage power generation circuit 215 and its connection to the HV driver 217.

The HV driver 217 includes a circuit having four PMOS transistors 509, 511, 513, 515 connected in series, specifically a first PMOS transistor 509, a second PMOS transistor 511 with a source terminal connected to a drain terminal of the first PMOS transistor 509, a third PMOS transistor 513 with a source terminal connected to a drain terminal of the second PMOS transistor 511, and a fourth PMOS transistor 515 with a source terminal connected to a drain terminal of the third PMOS transistor 513. The circuit further includes four NMOS transistors 517, 519, 521, 523 connected in series, specifically a first NMOS transistor 517 with a drain terminal connected to the drain terminal of the fourth PMOS transistor 515, a second NMOS transistor 519 with a drain terminal connected to a source terminal of the first NMOS transistor 517, a third NMOS transistor 521 with a drain terminal connected to a source terminal of the second NMOS transistor 519, and a fourth NMOS transistor 523 with a drain terminal connected to a source terminal of the third transistor 521, the fourth NMOS transistor 523 further having a drain terminal connected to ground.

The circuit also includes two further NMOS transistors 525, 527 connected in series, specifically a fifth NMOS transistor 525, and a sixth NMOS transistor 527 with a source terminal connected to a drain terminal of the fifth NMOS transistor 525; and two further PMOS transistors 529, 531 connected in series, specifically a fifth PMOS transistor 529 with a drain terminal connected to a drain terminal of the sixth NMOS transistor 527, and a sixth PMOS transistor 531 with a drain terminal connected to a source terminal of the fifth PMOS transistor 529.

A source terminal of the first PMOS transistor 509 is coupled to the output of the third voltage multiplier 405. A gate terminal of the first PMOS transistor 509 is coupled to the fourth clock signal CLK₄. A gate terminal of the second PMOS transistor 511 is coupled to an output of the second voltage multiplier 403 and a source terminal of the fifth NMOS transistor 525. The drain terminal of the second PMOS transistor 511 and the source terminal of the third PMOS transistor 513 are coupled to a gate terminal of the fifth NMOS transistor. A gate terminal of the third PMOS transistor 513 is coupled to the third non-overlapping clock signal C̅L̅K̅₃̅, the drain terminal of the fifth NMOS transistor 525 and the source terminal of the sixth NMOS transistor 527. The drain terminal of the third PMOS transistor 513 and the source terminal of the fourth PMOS transistor 515 are coupled to a gate terminal of the sixth NMOS transistor 527. A gate terminal of the fourth PMOS transistor 515 is coupled to the drain terminal of the sixth NMOS transistor 527 and the drain terminal of the fifth PMOS transistor 529. The drain terminals of the fourth PMOS transistor 515 and the first NMOS transistor 517 are coupled to a driver output 533. A gate terminal of the first NMOS transistor 517 is coupled to the drain terminal of the sixth NMOS transistor 527 and the drain terminal of the fifth PMOS transistor 529. The source terminal of the first NMOS transistor 517 and the drain terminal of the second NMOS transistor 519 are coupled to a gate terminal of the fifth PMOS transistor 529. A gate terminal of the second NMOS transistor 519 is coupled to the second non-overlapping clock signal C̅L̅K̅₂̅, the source terminal of the fifth PMOS transistor 529 and the drain terminal of the sixth PMOS transistor 531. The source terminal of the second NMOS transistor 519 and the drain terminal of the third NMOS transistor 521 are coupled to a gate of the sixth PMOS transistor 531. A gate terminal of the third NMOS transistor 521 is coupled to the input to the first voltage multiplier 401 and a source terminal of the sixth PMOS transistor 531. A gate terminal of the fourth NMOS transistor 523 is coupled to the second identity gate 415, i.e. the first clock signal CLK₁.

It should be appreciated that with the arrangement of FIG. 4 and FIG. 5, the three cascaded voltage multipliers 401, 403, 405 generate the clock signals and biasing at corresponding voltage domains, which directly control the output transistors to generate the square wave with an up to approximately 4V_{DD} voltage swing.

In the described embodiment, the circuit employs an input clock of 1V_{DD} swing from V_{DD} to circuit ground. In the described embodiment the Cₚᵤₘₚ of each stage, i.e. the capacitance of the six capacitors 418, 419, 421, 427, 425, and 423 is at least 20 times larger than the HV driver gate capacitance, therefore enabling the required drivability from the power transmitter 201 to be directly provided by the power output module 205.

FIG. 6 illustrates the dual-source power converter 317 and the power evaluation and feedback controller 319 in more detail.

The dual-source power converter 317 comprises a switch matrix 601, a pulse generation module 603, a pulse width controller 605 which includes a Maximum Power Point (MPPT) control module and a Zero Current Switching (ZCS) control module, a module 607 including control switch drivers and adaptive level shifters (ALS) and a plurality of control modules 605. Specifically, the dual-source power converter 317 comprises two ALS modules as will be apparent from the below.

The switch matrix 601 includes a transmitted power input from the power transmission rectifier 305a via a CMOS bilateral switch 609, and a harvested power input from the energy harvesting rectifier 305b via a first PMOS switch 611; an inductor 613 coupled in series to both the CMOS bilateral switch 609 and the first PMOS switch 611; two NMOS switches 615, 617 connecting either side of the inductor 613 to ground; a power evaluation circuit comprising a further connection to ground on the output side of the inductor 613 via a second PMOS switch 619 and a power evaluation capacitor 621 with a capacitor reset switch 623 connected across the capacitor 621; and a third PMOS switch 625 via which the inductor 613 supplies the load 627 of the wearable device.

In operation, the switch matrix 601 receives input voltage output by the two rectifiers 305a, 305b and converts it to the supply voltage V_{REG} for supplying the load of the wearable device via a first path including the third PMOS switch 625, i.e. power recovery from both the transmitted power and the harvested power from the ambient electromagnetic waves is integrated in the dual-source power converter 317. V_{REG} is regulated to a constant 1.1V, which is the nominal supply voltage for standard MOSFETS in 40 nm.

In addition, the switch matrix 601 defines a secondary charging path, via the second PMOS switch 619 via which power evaluation is performed. Thus, by means of the switch matrix 601, the dual-source converter is operable in both a load regulation mode and a power evaluation mode.

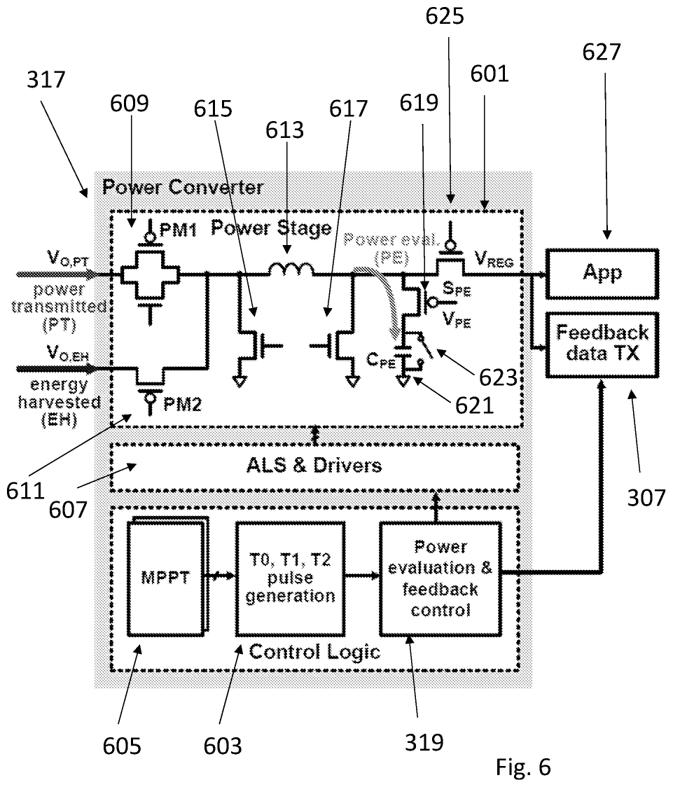

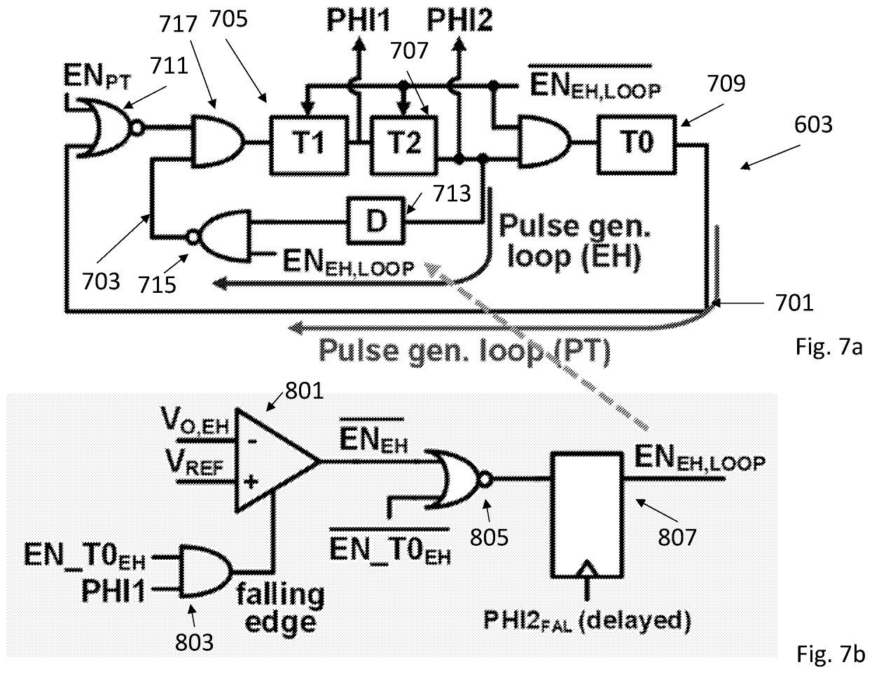

The pulse generation module 603 is illustrated in more detail in FIG. 7 and comprises an outer power transfer pulse generation loop 701 and an inner energy harvesting pulse generation loop 703.

The outer power transfer pulse generation loop 701 includes a tuneable T1 delay module 705, a tuneable T2 delay module 707, an AND gate with the delay from the T1 and T2 delay modules 705, 707, a tuneable TO delay module 709, and a first NOR gate 711 with the total delay from all three delay modules 705, 707, 709 and an enabling signal EN_{PT} as inputs with EN_{PT} held to zero when half the output voltage VREG is below a reference voltage. The enabling signal E̅N̅_̅{̅E̅H̅,̅L̅O̅O̅P̅}̅ is also provided to the tuneable T1 delay module 705 and the tuneable T2 delay module 707. It should be appreciated that for power transfer and harvesting, the T1 and T2 are different and the E̅N̅_̅{̅E̅H̅,̅L̅O̅O̅P̅}̅ signal determines which T1 and T2 times to employ as appropriate.

The inner energy harvesting pulse generation loop 703 includes the T1 delay module 705, the T2 delay module 707, a fixed delay module 713 and a second NOR gate 715 with the total delay from the three corresponding delay modules 705, 707, 713 and the inner loop enabling signal E̅N̅_̅{̅E̅H̅,̅L̅O̅O̅P̅}̅ as inputs.

The outputs of the NAND gate 715 and the NOR gate 711 are coupled via a second AND gate 717 which connects the two loops.

An output signal PHI1 is output following the T1 delay module 705 (which is common to both loops) to the module 607 including the control switch drivers and the adaptive level shifters (ALS) via the power evaluation and feedback controller 319 for control of the switch matrix 601 and resulting in charging of the inductor 613. Specifically, two ALS modules are included in the dual-source power converter 317: a first ALS module to generate the gating signal PM1 for control of the CMOS bilateral switch 609 and a second ALS module to generate the gating signal PM2 for control of the first PMOS switch 611.

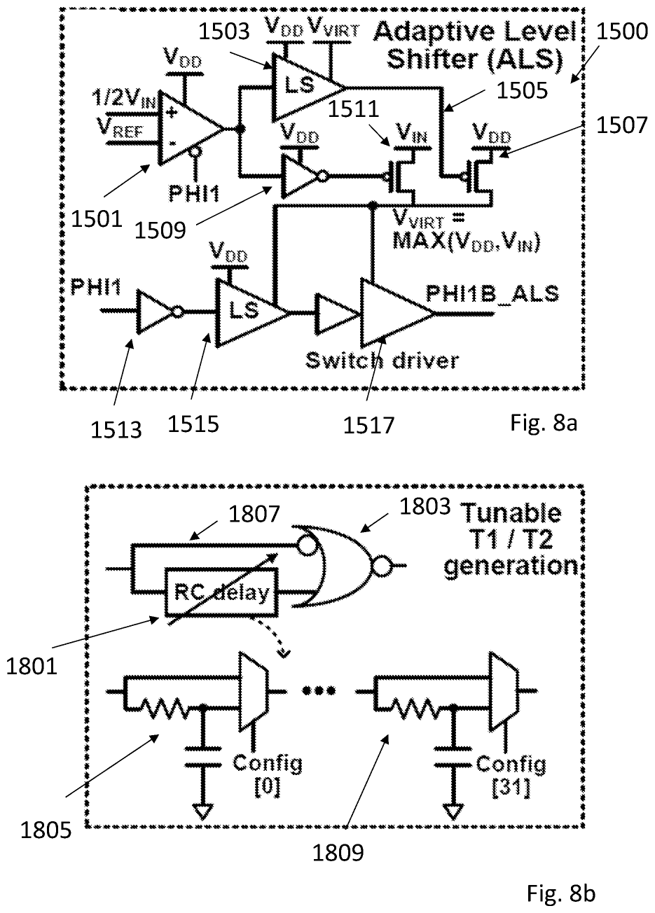

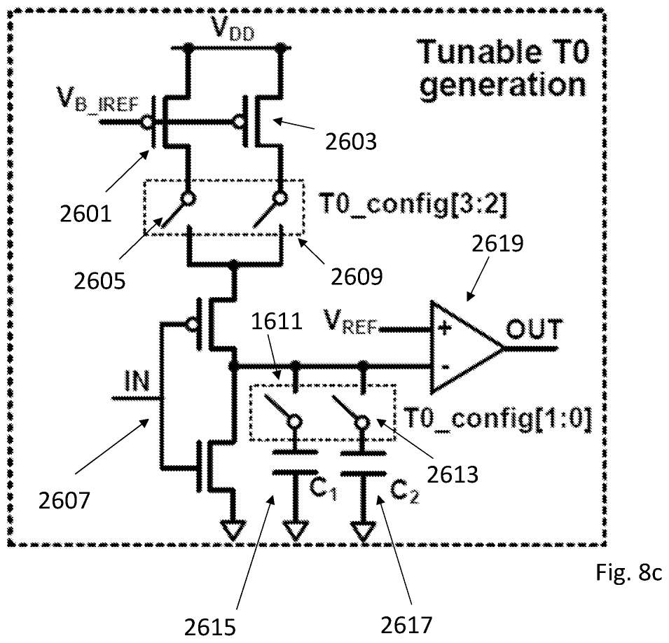

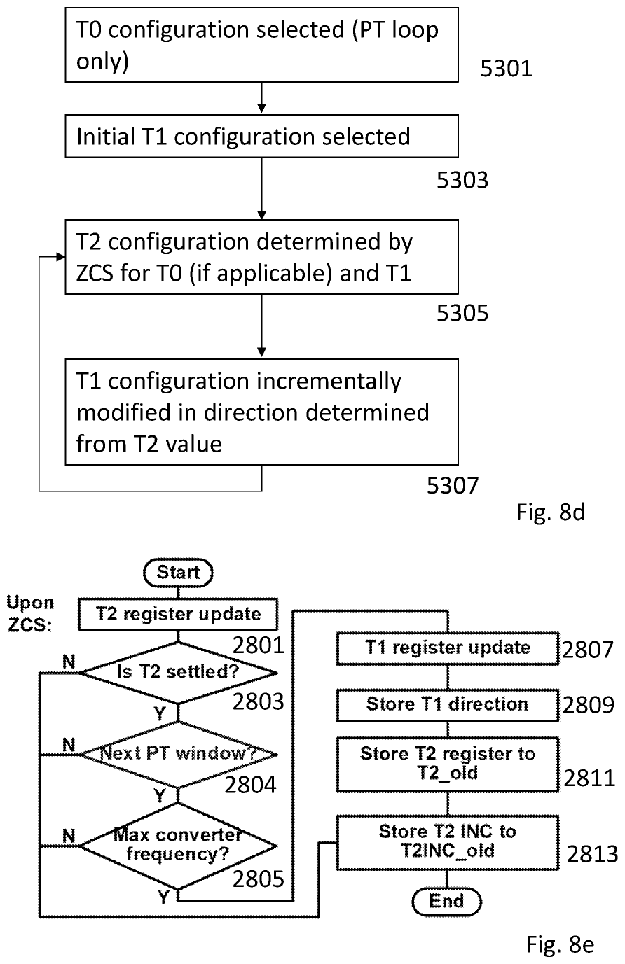

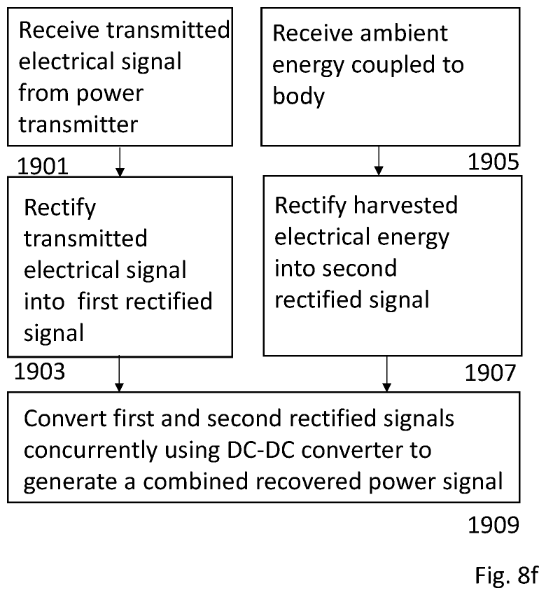

FIG. 8a schematically illustrates an exemplary one of the ALS modules 1500 (in the described embodiment, both ALS modules have the same arrangement) and switch driver which employ the signal PHI1 as inputs.

The ALS module 1500 comprises a dynamic comparator 1501 which compares half V_{IN}, the input voltage of the converter 309, against a reference voltage V_{REF} with V_{DD} as its supply and PHI1 as the clock. For the ALS module for PM1 generation, V_{IN} is V_{O,PT} and for the ALS module for PM2 generation, V_{IN} is V_{O,EH}. The dynamic comparator 1501 outputs to a level shifter 1503 and a NOT gate 1509 which are arranged in parallel. The level shifter 1503 has V_{DD} and V_{VIRT} as the low and high supplies, and the output 1505 of the level shifter 1503 is applied as a gate voltage to p-well transistor 1507 with supply voltage V_{DD}. The NOT gate 1509 has V_{DD} as the supply and its output signal is applied as gate voltage to p-well transistor 1511, with supply voltage V_{IN}.

The generation of the gate control signal PHI1B_ALS has a NOT gate 1513 taking the PHI1 signal as input, a level shifter 1515 and the switch driver 1307 giving output PH1B_ALS which is PM1 or PM2, depending the ALS module. Both level shifter and the switch driver shown in FIG. 8a take the maximum V_{VIRT} of V_{DD} and V_{IN} as the supply, V_{VIRT} being determined by ALS 1309. Level shifter 1515 also takes V_{DD} as a lower supply.

In operation, the dynamic comparator 1501 triggered by the PHI1 falling edge compares half V_{IN} against a reference voltage 1.1 V. V_{VIRT} takes on the higher potential between V_{IN} and V_{DD}, and is used as the supply PHI1 generation. The level-shifter 1503 turns off the header 1507 when V_{VIRT} is higher than the threshold voltage. Several start-up techniques are known in the art (see, for example, L. Lin, S. Jain and M. Alioto, “A 595 pW 14pJ/Cycle microcontroller with dual-mode standard cells and self-startup for battery-indifferent distributed sensing,” IEEE International Solid-State Circuits Conference (ISSCC) Dig. Tech. Papers, vol. 61, pp. 44-46, February 2018).

Returning now to FIG. 7a, an output signal PHI2 is also output following the T2 delay module (which is common to both loops) for control of the switch matrix 601 and resulting in discharging of the inductor 613. Thus, T1 and T2 correspond to the inductor 613 charging and discharging time, respectively. T0 corresponds to a discontinuous mode (DCM) time of the outer power transfer pulse generation loop 701.

FIG. 8b schematically illustrates the T1 delay module 705 and the T2 delay modules 707 of the pulse generation module 603 according to the described embodiment (both modules have the same arrangement). The two delay modules 705, 707 each comprise a tuneable RC delay line 1801 in parallel with a direct connection 1807 providing inputs to NOR gate 1803 with the input from direct connection 1807 inverted. Tuning of the RC delay line 1801 enables 32 configurations ([0] to [31]) of T1/T2 to be obtained, with exemplary configuration [0] 1805 and configuration 1809 shown in FIG. 8b.

FIG. 8c schematically illustrates the tuneable T0 delay module 703. The T0 delay module 703 comprises a CMOS inverter 2607 (having n-well transistor and p-well transistors connected in series) connected to V_{DD} via a circuit comprising a pair of p-well transistors 2601 and 2603 connected in parallel via respective switches 2605 and 2609 and both gated by V_{B_IREF}, which is 0.6V in the preferred embodiment. The output of the CMOS inverter 1607 is connected to static comparator 1619 which compares it with reference voltage V_{REF}.

The output of the CMOS inverter is also connected to parallel capacitors 2615, 2617 via switches 2611 and 2613, respectively.

In operation, the four switches 2605, 2609, 2611 and 2613 enable four configurations of the T0 delay module 703 (configurations 1 to 4) and thereby four selectable values of T0 1703. In the described embodiment, the selection depends on the V_{O,PT} level. Pre-defined V_{O,PT} levels are set, each range corresponding to a T0 selection.

The inner loop enable signal EN_{EH,LOOP} generation circuit is illustrated in FIG. 7b. The circuit comprises a dynamic comparator 801 which compares V_{REF} against the ambient energy received from the energy harvesting rectifier 305b triggered by an AND gate with an EH maximum power point (MPPT) condition EN_TO_{EH} and the PHI1 falling edge as inputs. The output of the dynamic comparator 801 is the inverse of an EH enable signal E̅N̅_̅{̅E̅H̅}̅ and is input into a NOR gate 805 which additionally has E̅N̅_̅T̅O̅_̅{̅E̅H̅}̅, the inverse of the EH maximum power point condition EN_TO_{EH} as an input. The NOR gate 805 outputs to a module 807 which outputs EN_{EH,LOOP} upon a delayed PHI2 falling edge which is delayed by a standard RC (resistor-capacitor) delay circuit (not shown).

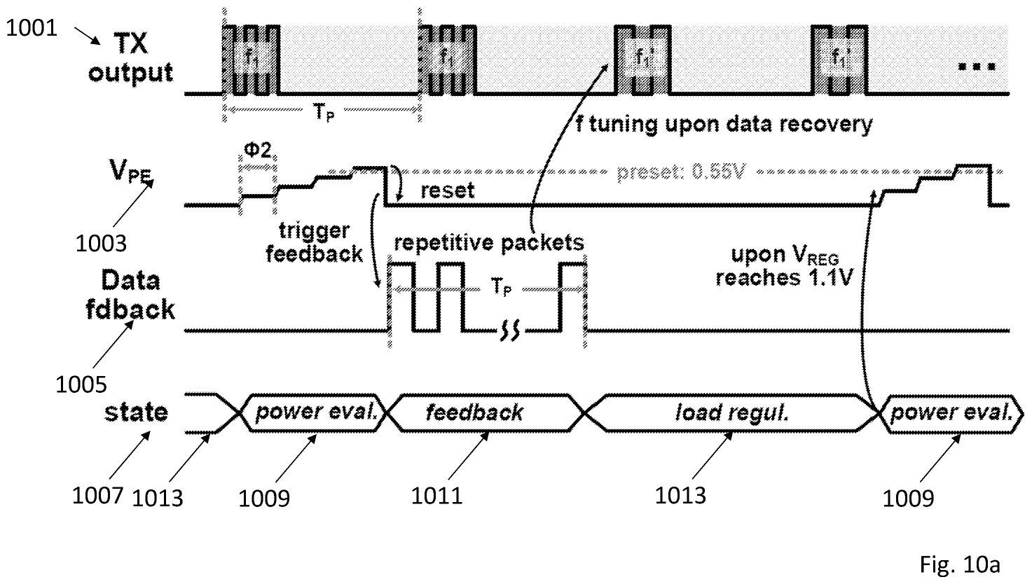

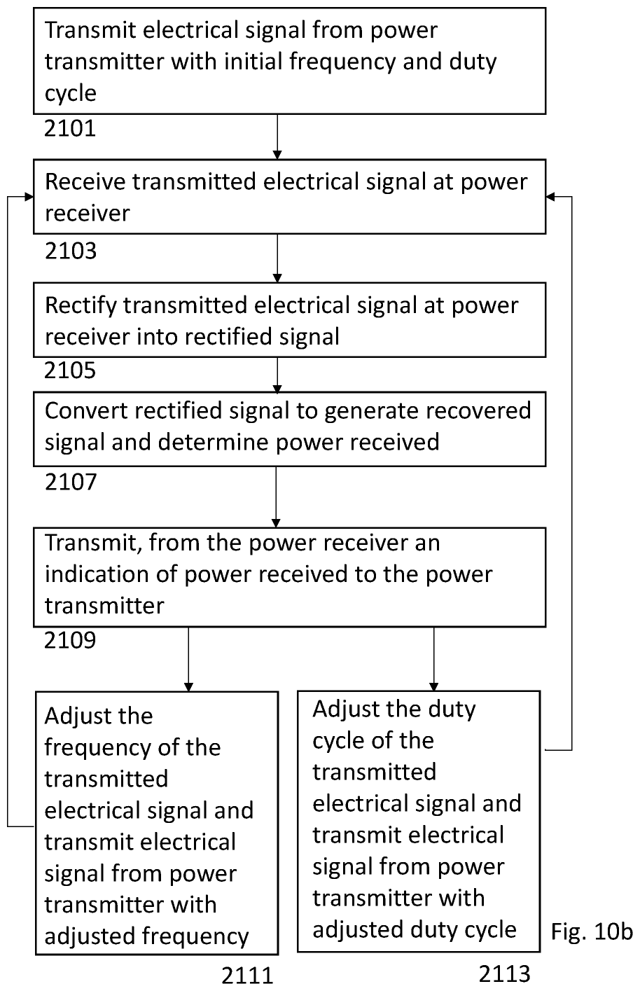

In operation, as will be discussed below (see e.g. FIG. 10a), the power transmitter 203 transmits power over a period Tₚ, i.e. power is transmitted in distinct power transfer windows. Power recovered is delivered to the load V_{REG} to meet the RX power demand at a regulated voltage (1.1V in the described embodiment). Here, the dual-source power scavenging (i.e. both via power transmission and energy harvesting) is performed in an asynchronous manner to suppress quiescent current. The T0 delay module 709 determines the minimum T0 based on V_{O,PT} (and V_{O,EH}) level—the pre-defined V_{O,PT} (V_{O,EH}) levels determine the configuration of T0 module, as discussed above. An actual discontinuous conduction mode (DCM) time is determined asynchronously by V_{REG} level (i.e. comparison with 1.1 V). The inductor charging time (T1) is determined by maximum power point tracking (MPPT) by the MPPT control module of the pulse width controller 605 to enable an input power of the dual-source converter to reach a maximum value, whereas the inductor discharging time (T2) is determined by zero-current switching (ZCS) using the ZCS control module of the pulse width controller 605 (see L. Lin, S. Jain and M. Alioto, “Integrated Power Management for Battery-Indifferent Systems With Ultra-Wide Adaptation Down to nW,” IEEE Journal of Solid-State Circuits, vol. 55, no. 4, pp. 967-976, April 2020 and D. El-Damak and A. P. Chandrakasan, “A 10 nW-1 μW Power Management IC With Integrated Battery Management and Self-Startup for Energy Harvesting Applications,” IEEE Journal of Solid-State Circuits, vol. 51, no. 4, pp. 943-954, April 2016 for a description of ZCS).

FIG. 8d shows an outline of the method of determining T1 and T2. In step 5301, the configuration of the T0 delay module 703 is selected in the PT loop. Note that this step is omitted in the EH loop as there is no T0 module in the inner loop 703. In step 5303 (in either loop), an initial configuration for the T1 delay module 705 is selected. In step 5305, the ZCS configuration of the T2 delay module 707 is determined given the selected configuration of the T0 709 delay module (if applicable) and the T1 delay module 705. In 5307, the configuration of the T1 delay module is altered to an adjacent configuration. The direction of adjustment of the configuration of the T1 delay module 705 is determined by the trend of change in the value of the inductor discharging time (T2) which represents the input power trend of change, as will be described below. The method then returns to step 5305.

FIG. 8e illustrates the method of asynchronous MPPT performed to determine T1 (which is dictated by the configuration of the T1 delay module 705), i.e. the method performed in step 5307 of FIG. 8d. Upon T2 being updated by ZCS in step 5305, the T2 register is updated in step 2801 and the completion status of T2 adjustment under a given T1 is determined in step 2803. If the T2 is settled, then, in step 2805 it is determined if the converter 309 is operating at the maximum frequency f_{sw}. Maximum f_{sw} is reached when the pulse generation loop is re-triggered after the T0 module completes. In the described embodiment, a detector circuit is implemented to determine the EN_{PT} (or EN_{EH}) status at the instance of T0 module completion. Note that in the case of power transfer, there is an additional step 2804 prior to step 2805 to determine that a new power transfer window has been started. This is because, in the case of power transfer (in contrast to energy harvesting), only one T1 adjustment per power transfer window is performed. If all requirements in steps 2803-2805 are met, as appropriate, in step 2807 the T1 register is updated, i.e. it is shifted by one bit in the direction determined in 5307, and in step 2809, the currently settled T2 is compared with the stored value and the T1 increment/decrement direction is determined and stored. If the T2 settled value is larger than the earlier settled value, T1 is incrementally adjusted in the same direction as in the previous adjustment. Otherwise, T1 adjusted in the opposite direction. At the very start of the process, T1 is incrementally increased, introducing perturbation for T2 change, which then affects the T1 direction later on. In step 2811 the current T2 value is stored, and in step 2813, the ZCS decision is stored, i.e. the incremental change in T2.

It will be appreciated from FIG. 8e and the above description that while, for energy harvesting, the T1 is adjusted for each update in T2, in the case of power transfer, T1 is no longer adjusted within each power transfer window where the general trend perturbs T2 indication. Instead, T1 adjustment (i.e. MPPT) is enabled in a slower manner, with one T2 feedback and thus one T1 adjustment per power transfer window. To ensure consistent and fair power level indication across different power transfer windows, in the described embodiment the T2 value of each power transfer window is taken when the settled T2 value changes from an increasing trend to a decreasing trend. With a fixed T1 and thus fixed converter input impedance, this turning point is solely due to the time division multiplex-based transmission (see below for detailed discussion) and is thus used to represent each power transfer window. As shown in FIG. 8e, by comparing the settled T2 values taken at each power transfer window, the direction of T1 adjustment is determined, where an increase in T2 suggests to maintain the current T1 register update direction at the next power transfer window, and a decrease in T2 suggests otherwise.

It should be appreciated from FIGS. 7a and 7b that the pulses for switch control are generated asynchronously. The outer loop generates control signals for transmitted power recovery. It self-oscillates upon the output falling below 1.1V. When sufficient ambient energy is accumulated and when the MPPT condition is met, the loop enable signal (EN_{EH,LOOP}) will be asserted upon a delayed PHI2 falling edge. The inner loop then generates control signals to recover harvested power. In this inner EH loop, the delay module which delays the PHI2 falling edge and the delay module 713 are inserted to allow timely T1 and T2 register update. The EH enable signal EN_{EH} is pulled down upon inner loop PHI1, and the loop enable signal EN_{EH,LOOP} is pulled down upon inner loop PHI2, to avoid double assertion.

Thus, the DC-DC converter integrates power recovery from both the transmitted power and the harvested power from the ambient electromagnetic waves at the maximum power point of each source.

FIG. 8f summarises the method of power recovery at the first power receiver 301a according to the described embodiment.

In step 1901, a transmitted electrical signal is received from the power transmitter 201 at the electrode 311.

In step 1903, the transmitted electrical signal is rectified at the power transmission rectifier 305a.

In step 1905, ambient energy coupled to the body is received at the electrode 311.

In step 1907, the ambient energy is rectified at the energy harvesting rectifier 305b.

In step 1909, both the rectified transmitted power and rectified harvested energy are concurrently converted at the dual-source converter 317 to generate a combined recovered power signal to supply the load of the wearable device.

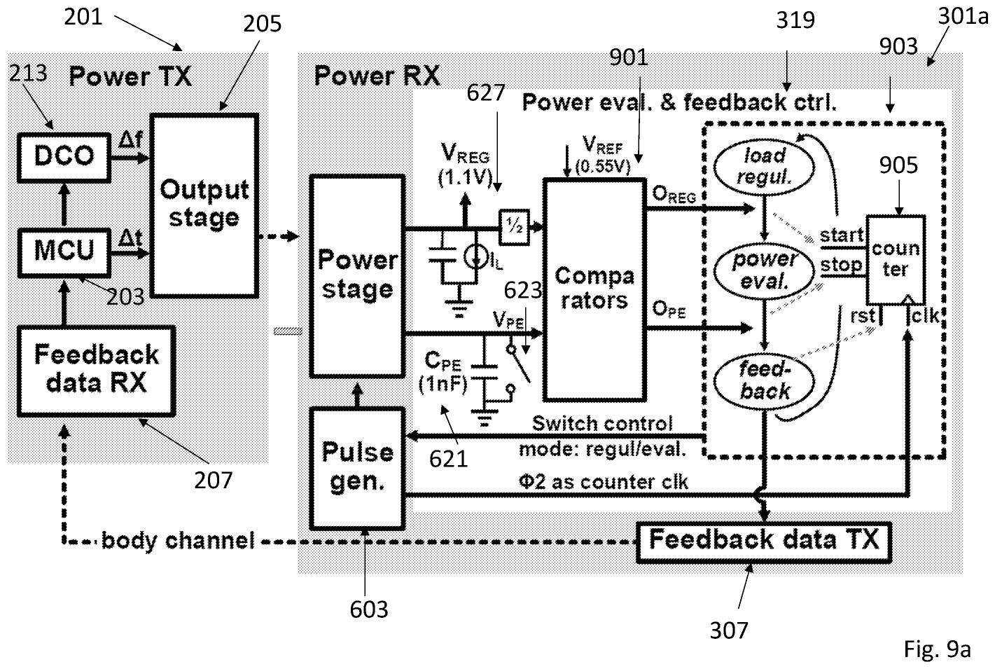

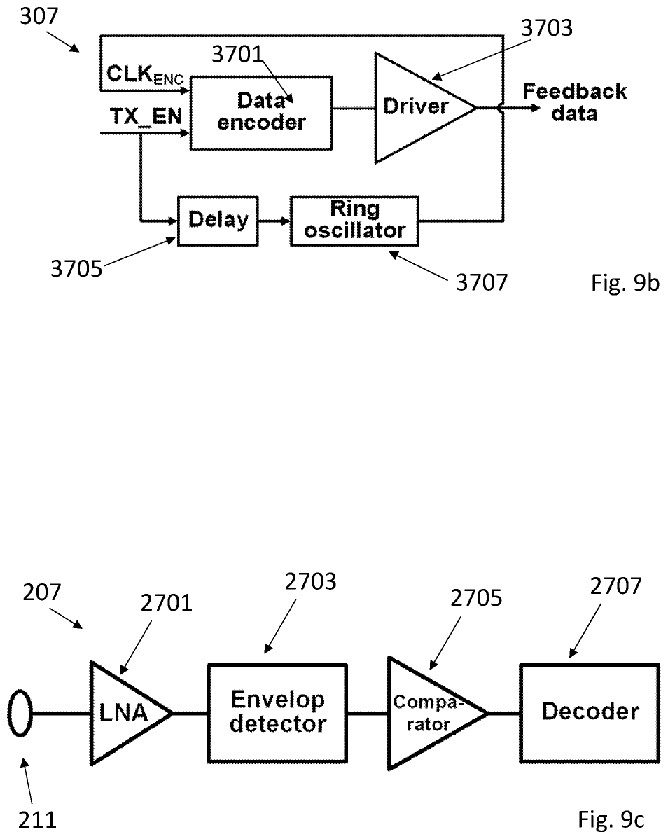

The power evaluation and feedback control module 319 and its operation are schematically illustrated in FIG. 9a.

The power evaluation and feedback control module 319 includes a comparator module 901 and a state machine 903 including a counter 905. The comparator module 319 is configured to receive the voltage across the power evaluation capacitor 621 and half of the supply voltage V_{REG} as inputs and to compare each of them when received with a reference voltage which, in the described embodiment is 0.55V. The comparator module 901 is configured to output corresponding comparison results to the state machine 903. The state machine 903 is configured to cycle between three operating states based on the output of the comparator module 901, specifically load regulation, power evaluation and feedback. The state machine 903 is configured to output control instructions to the pulse generation module 603 based on a current operating state to control the switches 609, 611, 615, 617, 619, 625 of the switch matrix 601. The counter 905 also receives input from the pulse generation module as will be explained below. In the feedback state, the state machine 903 outputs feedback data to the data transmitter 307 for transmission via the body channel to the data receiver 207 of the power transmitter 201.

It should be appreciated that the state machine may implemented in digital circuits according to conventional techniques.

FIGS. 9b and 9c illustrate the data transmitter 307 and data receiver 207 according to the described embodiment, respectively.

As illustrated in FIG. 9b, the data transmitter 307 includes a data encoder, a driver 3703, a delay block 3705 and ring oscillator 3707. The data encoder outputs to the driver 3703 for transmission of the feedback data upon assertion of both TX_EN and CLK_{ENC} signals. During the feedback state, when the data packets are ready, TX_EN is asserted to enable the data encoder 3701 and also the ring oscillator 3707 via the delay block 3705, the right oscillator 3707 itself asserting CLK_{ENC}. Thus, the delay block 3707, which is inserted before the ring oscillator is enabled by TX_EN, ensures that the data encoding completes before CLK_{ENC} triggers the serialized data output.

As illustrated in FIG. 9c, in the described embodiment, the data receiver 207 comprises non-coherent data receiver structure, including a low-noise amplifier (LNA) 2701, an envelope detector 2703, a comparator 2705, and a decoder 2707 connected in series.

FIG. 10a illustrates the operation of power transfer between the power transmitter 201 and the first power receiver 301a and schematically shows the power output 1001 to the first power receiver 301a of the power transmitter 201, the voltage V_{PE} 1003 across the power evaluation capacitor 621, the data 1005 transmitted by the data transmitter 307, and the state 1007 of the state machine 903.

Upon initiation, the state machine 903 is in the load regulation state 1013 to ensure there is sufficient energy available at RX to support the following evaluation and feedback operation. Upon V_{REG} reaching 1.1V, the state machine 903 transitions to the power evaluation state 1009. In the power evaluation state 1009, the power transfer path to V_{REG} is disabled via opening of the third PMOS switch 625 and is redirected to the power evaluation capacitor C_{PE} 621 by closing the of second PMOS switch 619. To evaluate only the transmitted power, the first PMOS switch 611 is also opened, thereby disabling energy harvesting. For a consistent power level evaluation, the T0 is also forced at its minimum (i.e. conversion frequency forced at the maximum).

The power transmitter 203 transmits power at an initial frequency, duty cycle and period Tₚ which, when, received by the first power receiver causes a stepwise increase in voltage across the power evaluation capacitor 621, each step being determined by the inductor discharging time T2 (also called φ₂). The counter 905 tracks the number of conversion cycles taken for V_{PE} to reach the preset threshold (as determined by the comparator module 901) which, in the described embodiment is 0.55V.

Once V_{PE} reaches or exceeds the preset threshold, the state machine 903 transitions to the feedback state 1011, where data preparation is performed and feedback data is transmitted over the body channel from the data transmitter 307. During the feedback state, power recovery is disabled to avoid conflicts in the body channel which is shared by both power transmission and data feedback. Both the counter 905 and the power evaluation capacitor C_{PE} 621 are reset in this state. The feedback data is transmitted in the form of repetitive packets which are received at the power transmitter 201 data receiver 207 and comprises details regarding the number of cycles taken for V_{PE} to reach the preset threshold, which it should be appreciated is inversely proportional to the transmitted power recovered. Additionally, the feedback data comprises details of a frequency setting of the first receiver 301a (which is determined by the variable capacitor 321).

Based on the feedback received, the power transmitter 201, specifically the MCU 203 causes an adjustment in the frequency and duty cycle of the digitally controlled oscillator 213 for its power output 1001 to the first power receiver 301a to optimize the power transmitted to the first power receiver 301a. Specifically, the frequency is tuned first to optimize for both the specific user's body 105 and also the path to the first receiver 301a, i.e. the specific position of the first power receiver 301a relative to the position of the power transmitter 201. Following tuning of the frequency, the duty cycle for transmission at the particular frequency for transmission to the first power receiver 301a is adjusted to control the power transmitted in order that the recovered power at the first receiver 301a approximately matches the power requested by the relevant wearable device. In the described embodiment, the requested power for each wearable device is pre-stored at the power transmitter 201.

Once the state machine 903 has been in the feedback state 1011 for a time period equal to the initial period of the power transmitter 203 Tₚ (which may ensure that the power transmitter 201 receives the data packets in spite of potential clashes with the transmitted power signal in the channel), the state machine 903 automatically transitions to the load regulation state 1013 in which both energy harvesting and power transmission are enabled and energy evaluation is disabled. Thus, it should be appreciated that the data transmitter is only enabled upon the completion of power evaluation, and is self-disabled upon one transmission cycle.

Once the supply voltage V_{REG} reaches the predetermined supply voltage (1.1V in the described embodiment), the state machine 903 transitions back to the power evaluation state 1009, and the cycle repeats.

In the described embodiment, the frequency of the digitally controlled oscillator (to control frequency at the power transmitter 201) as well as the variable capacitor 321 (to control frequency at the first receiver 301a) are initially adjusted as a one-time process to optimize for subject/path specificity, i.e. the optimal frequency for transmission to the particular receiver is determined in this cycle. For example, five frequencies are preset at both the power transmitter 201 and the first power receiver 301a. Starting from the first preset frequency setting for the first power receiver 301a, the first power receiver 301a is configured to evaluate the power received and then sends the feedback data to the power transmitter 201 as described above. The first power receiver 301a subsequently transitions to the second frequency setting for the first power receiver 301a (by adjusting the variable capacitor 321) at the completion of the feedback state 1011, and the power transmitter 201 transitions to the next frequency setting (by adjusting the frequency of the DCO) after receiving and decoding the data (which contains the current power receiver frequency setting information, as discussed above), until the fifth frequency setting has been evaluated. Both the power transmitter 201 and the first power receiver 301a then then select the frequency setting that results in the highest evaluated power. In the described embodiment, the step size between each of the five frequency settings is approximately 2 MHz, with an overall operation range for the first power receiver spanning approximately 10 MHz.

Once the frequency adjustment has been performed, the duty cycle is then adjusted periodically in the subsequent cycles following each transition to the feedback state 1011. As discussed above, the duty cycle is adjusted such that the recovered power at the first receiver 301a approximately matches the power requested by the relevant wearable device.

FIG. 10b provides a summary of this process.

In step 2101, the power transmitter 201 transmits an electrical signal with an initial frequency and duty cycle.

In step 2103, the first power receiver 301a receives the transmitted electrical signal.

In step 2105, the transmitted electrical signal is rectified at the power transmission rectifier 305a.

In step 2107, the rectified signal is converted to generate a recovered signal and the power received is determined via the power evaluation circuit.

In step 2109, an indication of the power received is transmitted to the power transmitter 201.

In step 2111, the frequency of the transmitted electrical signal is adjusted at the power transmitter 201 and power is subsequently transmitted with the adjusted frequency. The process then returns to steps 2103-2111.

Once an optimal frequency has been determined (i.e. the frequency resulting in greatest power recovery at the receiver), following step 2109, the method proceeds to step 2113 and the duty cycle is adjusted. Again, the method then cycles through steps 2103-2109 and 2113, adjusting the duty cycle in step 2113 until an optimal duty cycle is determined (i.e. the minimum duty cycle for meeting the requested power of the corresponding wearable device). In the described embodiment the process of adjusting the duty cycle is performed periodically.

In more detail, at the first power receiver 301a, the state machine 903 controls the dual-source power converter 317 to perform regulation with maximum power extraction via the main conduction path, or power evaluation by charge accumulation along a secondary, power evaluation path. Upon completion of the evaluation, the data transmitter 307 encodes and transmits the power level information to the power transmitter 201 along the body channel. The output frequency and duty cycle are then adjusted accordingly at the power transmitter 201. The power transmitter 201 frequency adaptation (upon first initiation) and power adaptation (periodically) cycle, energy accumulation at the power evaluation capacitor 621 begins once the regulated voltage reaches 1.1V, with the pulse generation T2 (inductor 613 discharging time) as a clock to track the number of conversion cycles until the voltage across the power evaluation capacitor 621 reaches a preset threshold, which in the described embodiment is 0.55V. Inversely proportional to the power recovered, such number of conversion cycles is used to indicate the first power receiver 301a power level, which is then encoded in the average power being evaluated and multiple power transmitter 201 cycles for regulation.

It should be appreciated that this process is performed for each of the three power receivers 301a, 301b, 301c. As noted above, time dependent multiplexing is employed to transmit different frequencies to each of the three power receivers 301a, 301b, 301c and each of the three power receivers 301a, 301b, 301c is configured to perform frequency rejection so as to only recover power from the portion of the transmitted signal corresponding to itself.

Specifically, in the described embodiment, each of the three power receivers 301a, 301b, 301c is allocated to a frequency range spanning approximately 20 MHz, with the step size between each of the five frequency settings for each power receiver approximately 2 MHz, as noted above. The frequency ranges of the three power receivers 301a, 301b, 301c are also separated by approximately 20 MHz so that each of the three power receivers 301a, 301b, 301c has a distinct operating range, which may ensure that power is only recovered from the corresponding portion of the transmitted signal. For example, in the described embodiment the step size is approximately, 4 MHz and the first power receiver 301a operates at approximately 4 MHz˜20 MHz, the second power receiver 301b operates at approximately 40 MHz˜56 MHz, and the third power receiver operates at approximately 80 MHz˜96 MHz. In other variations, the frequencies of the power receives may range from approximately 1 MHz to approximately 100 MHz.

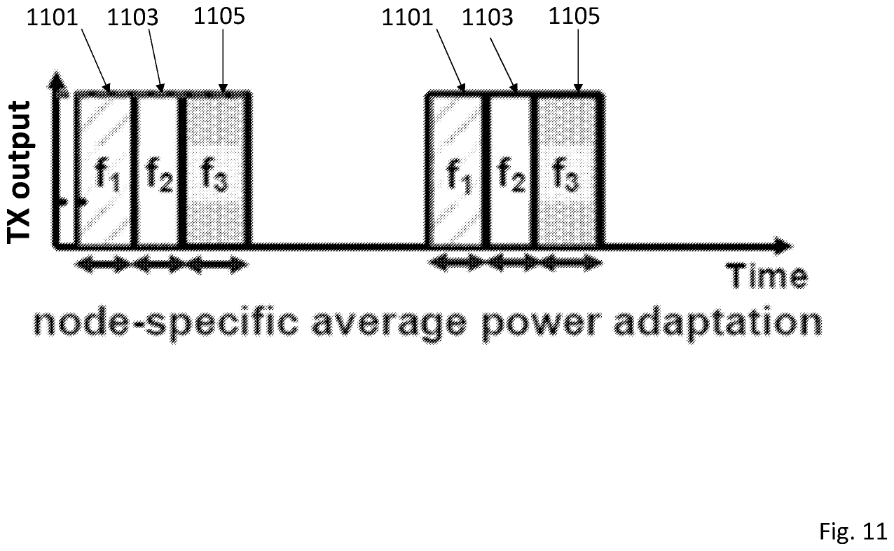

FIG. 11 illustrates an example of a total signal transmitted by the power transmitter 201 including three different frequency components 1101, 1103, 1105. Specifically, the total signal includes a first frequency component 1101 having a first frequency f₁ and first duty cycle adapted to the first power receiver 301a, a second frequency component 1103 having a second frequency f₂ and first duty cycle adapted to the second power receiver 301b, and a third frequency f₃ and third duty cycle adapted to the third power receiver 301c.

In summary, by recovering and interpreting the feedback data on the power received, the power transmitter 201 fine-tunes the transmission frequency and adjusts its output power for the three power receivers 301a, 301b, 301c. Meanwhile, the three power receivers 301a, 301b, 301c recover power from both the transmitted source and electromagnetic waves coupled onto body from the ambience. Power evaluation is also integrated into each of the three power receivers 301a, 301b, 301c, which perform periodic/event-driven estimation of the power recovered from the transmitted source. This information is then relayed to the power transmitter 201 by an event driven data transmitter 307 via the same body channel.

By delivering power to multiple wearable/implantable nodes via the human body, where its output power and frequency to each node are adapted based on the powering condition feedback by power receiver at each node the power transmitter 201 may 1) tailor its output frequency to the optimal frequency of each transmission path for highest powering efficiency; and 2) adapt its output power to deliver adequate power to each of the three power receivers 301a, 301b, 301c while avoiding wastage due to over-powering. In other words, the power transmitter 201 may be able to wirelessly deliver power to multiple wearable nodes while maintaining automatic and node-specific output power/frequency control, which may enable the power transmitter 201 to operate at optimal (i.e. lowest) energy consumption for prolonged battery lifetime.

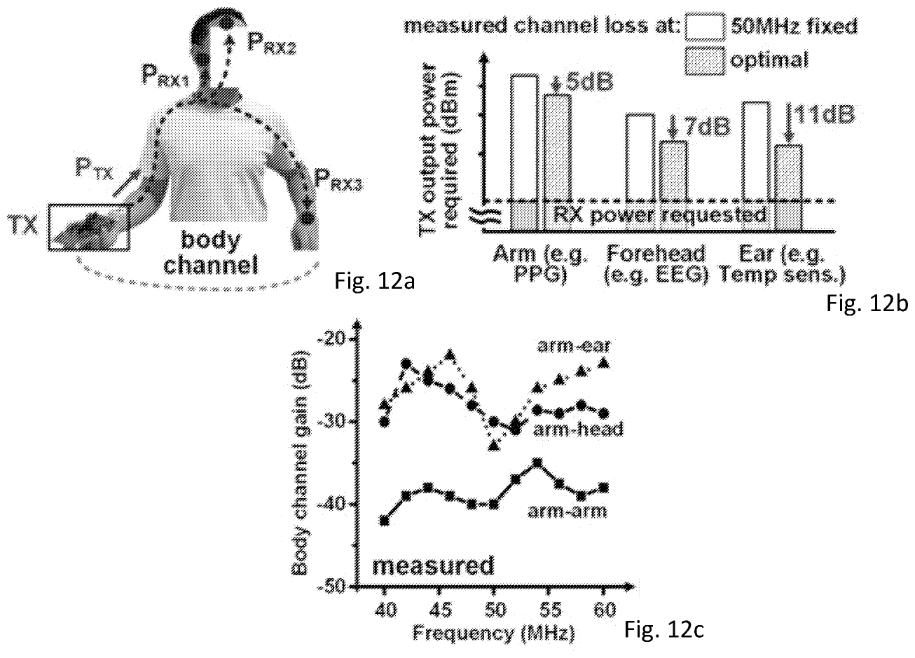

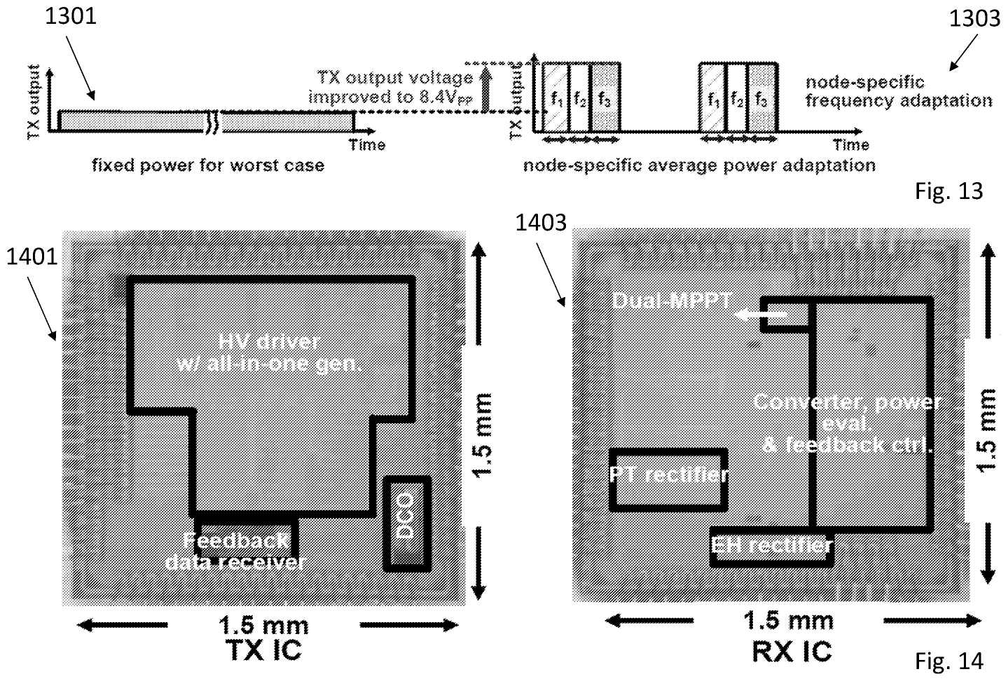

It should be appreciated that, without frequency and power adaptation, in order to avoid the worst-case scenario of power shortage, the power transmitter output power would always need to margin for the largest power demanded from the node of highest path loss (e.g. placed farthest away from the hub). One such scenario is illustrated in FIG. 12a-d which illustrate transmitter output power required (FIG. 12b, comparing power output required for a signal with an optimal frequency and a signal a fixed frequency of 50 MHz) and Body channel gain as a function of signal frequency (FIG. 12c) for three receivers positioned at different positions on a body (shown in FIG. 12a), specifically the left arm, the forehead and the ear and a transmitter positioned on the right wrist. As will be appreciated from FIG. 12c, using the human body as a power transmission medium, the optimal frequency varies along each path, which leads to 5-11 dB channel degradation when the TX frequency is fixed at 50 MHz, as may be appreciated from FIG. 12b. Meanwhile, accounting for the largest power demand results in wastage with the received power significantly larger than that required. FIG. 13 illustrates the fixed output 1301 of a TX in this scenario compared with the node-specific output 1303 according to the described embodiment.

Thus, the duty cycle and frequency adaptation of the described embodiment may overcome the path loss degradation at fixed frequency due to subject or path variations and ensure that adequate power is ensured at each node by transmitter output power adaptation, which, in the meantime, avoids transmitter power overdraw and thus prolongs transmitter battery lifetime. Further, this may ensure full-body coverage of power transmission, i.e. the receiver and transmitter may be placed at any location on the body.

In addition, it should be appreciated that the power output module 205 enables high voltage generation in a standard CMOS (Complementary Metal-Oxide-Semiconductor) process without requiring a level-shifter, biasing circuit or buffers in the generation of the high voltage swing. This may result in power reductions.

Further, concurrent recovery of the body-coupled transmitted power and the body-coupled harvested power by the dual-source power converter 317 may enable the two body-coupled sources to be recovered simultaneously at their respective maximum power point, improving the recovered power. Further, the two body-coupled sources may be able to share the same electrode, reducing device form factor.

Yet further, it will be appreciated that, in the described embodiment, power evaluation is integrated in the design of the dual-source power converter 317, thereby enabling the evaluation of actual recovered power. Further, average power is evaluated which does not require TX-RX synchronization, which may simplify the design of the energy transfer apparatus 100.

By employing asynchronous pulse generation and Maximum Power point tracking, the dual-source power converter 317 may further be insusceptible to the TDM-induced input power variation enabling power stability.

Conventional powering methods/apparatus for wearables require the removal of the device for wired (i.e. plugged into a supply) or wireless (i.e. put on a charging pad) charging, which constrains the user activity and disrupts the device operation. In contrast, the energy transfer apparatus 100 according to the described embodiment may ensure that a sufficient amount of power is delivered to the wearable devices in a wireless and on-body manner (i.e. device removal may no longer be required). Further, battery life-time of the powering source may be extended, by transmitting sufficient but not excessive power at the optimal transmission frequency.

It should be appreciated that the energy transfer apparatus 100 according to the described embodiment may offer an on-body wireless powering solution for a wide variety of applications including fitness tracking, health monitoring (e.g. blood glucose, electrocardiogram care, etc), assisted living, smart fabrics and gaming accessories.

A prototype power receiver 1403 and power transmitter 1401 were fabricated in 40 nm CMOS process according to the described embodiment, wherein the TRX ICs occupied an active area of 1.32 mm² (TX) and 0.84 mm² (RX). Die micrographs of the prototype power receiver 1403 and power transmitter 1401 are shown in FIG. 14.

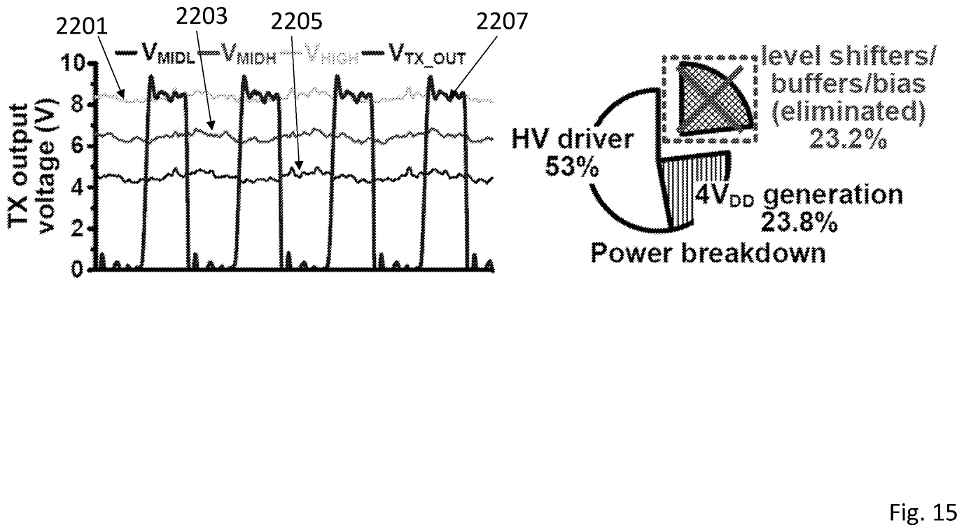

FIG. 15 shows the measurement results of the high voltage pulse generation at the prototype power transmitter 1401, showing corresponding outputs for the three cascaded voltage multipliers which are supplied to the HV driver (see FIG. 4), specifically V_{MIDL} 2201, V_{MIDH} 2203, V_{HIGH} 2203 and the output swing V_{TX_OUT} 2207. With a 2.5V input clock, the output swing V_{TX_OUT} 2207 was boosted to 8.4V under the standard CMOS process. By eliminating the level shifters, biasing, and tapered buffer circuits required conventionally, it is estimated that 23.2% power reduction is achieved when the TX operates at 70 MHz. The device reliability was ensured by maintaining the voltage across all the gate oxides within the nominal voltage, and by keeping the reverse bias across the well sufficiently below the breakdown limit.

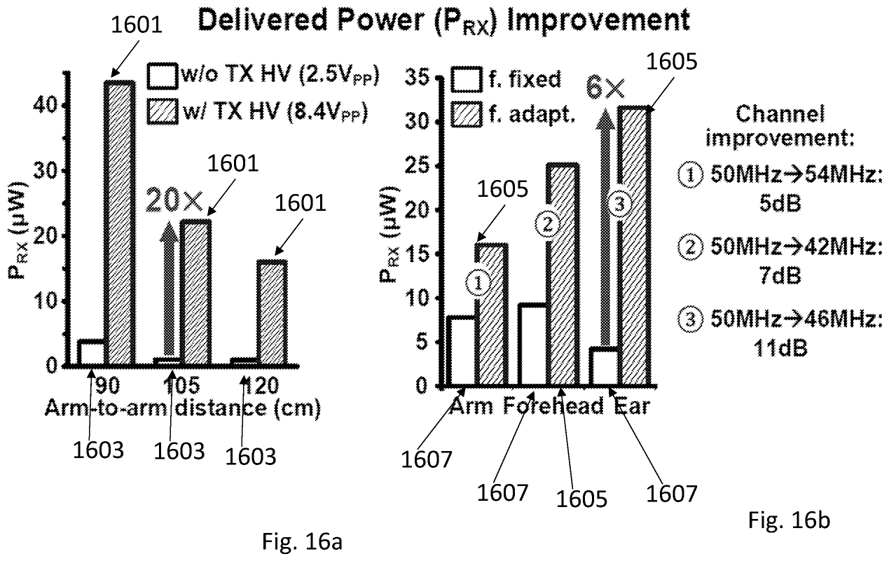

FIG. 16a illustrates the maximum power delivered using the TX prototype 1401 (bars 1601) with the compared with a conventional 2.5V_{PP} output (bars 1603). As will be appreciated from FIG. 16, the HV driver at the power transmitter according to the described embodiment improves the maximum delivered power by up to 20× (at 105 cm on-body distance), compared to a conventional 2.5V_{PP} output (bars 1603). The improvements over 90 cm and 120 cm (on-body) are 11× and 16×, respectively. Such differences could be attributed to the node-side power recovery, where the rectifier efficiency improves significantly when input signal voltage increases to above switch threshold.

FIG. 16b illustrates power delivered with frequency adaptation according to the described embodiment (bars 1605) compared with conventional fixed frequency power transmission (bars 1607) (using a frequency of 50 MHz). With the optimal transmission frequency swaying due to the heterogeneous and subject/path dependent channel characteristics, the frequency adaptation may improve the delivered power by adapting to the optimal channel. The measurement results of FIG. 16b show up to 6× higher delivered power (adapting to 46 MHz yielding 11 dB channel improvement), compared to the fixed frequency of 50 MHz. Conversely, this improvement factor corresponds to the amount of TX power saving for a given power request.

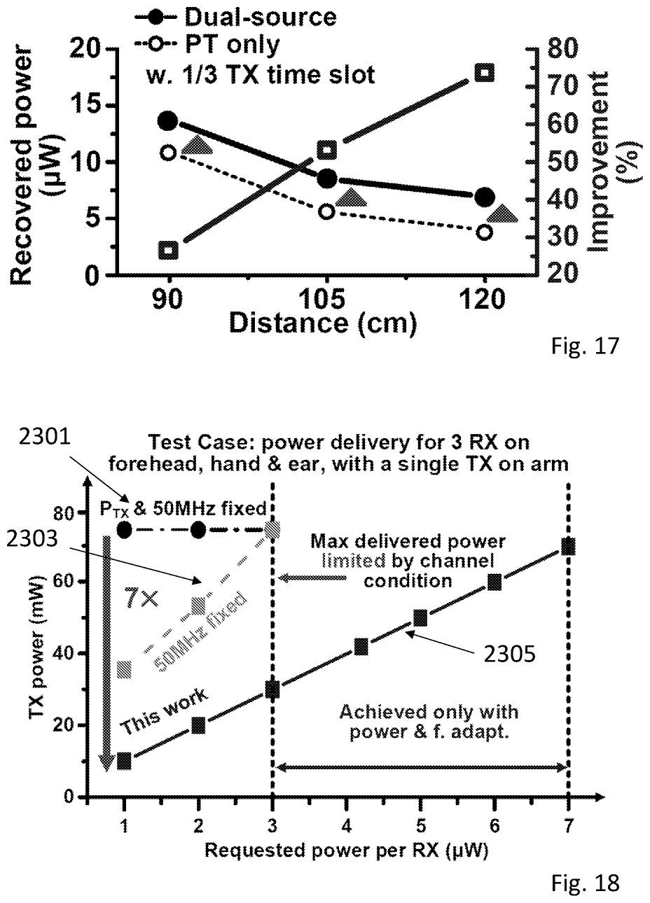

FIG. 17 illustrates percentage improvement in receiver power (right hand axis) due to dual-source power recovery compared with power transmission recovery with ⅓ TX output duty cycle, as a function of transmission distance. As will be appreciated from FIG. 17, the advantage of the concurrent and continuous scavenging of body-coupled ambient source becomes more prominent at longer transmission distance, where the power recovered from the transmitted source decreases. At 120 cm apart with ⅓ TX output duty cycle, the power recovered from the ambient electromagnetic waves improves the overall power recovery by 74%.

FIG. 18 illustrates results for a multi-node test case where a TX (placed on arm) delivers power to 3 RX (placed on forehead, hand and ear) of fixed power request ranging from 1 μW to 7 μW, showing transmitter power for fixed output power and frequency of 50 MHz (line 2301), fixed frequency of 50 MHz but variable TX power (line 2303) and for both frequency and power adaptation according the described embodiment (line 2305). Power and frequency adaptation according to the described embodiment (line 2305) was found to enable 7× TX power saving, considering a power request of 1 μW from all three RX nodes. Due to the constraints of the channel condition under 50 MHz fixed power delivery, power request from the RX placed at ear could no longer be satisfied when the power request increased above 3 μW. On the contrary, up to 7 μW per RX was satisfied with adaptation according to the described embodiment, doubling the amount achievable otherwise.

Lastly, it was found that under a 0.1 Hz adaptation cycle, the feedback circuits of the prototype apparatus consumed only ˜90 nW, thus the frequency and duty cycle adaption may be efficient.

In summary the energy transfer apparatus 100 according to the described embodiment provides an on-body powering system with node-specific channel and power condition awareness at the power transmitter 201. The frequency and power adaptation closes the loop for power transmission, which may enable the power transmitter 201 to sustain RX nodes placed around the human body without power overdraw, via a channel of optimal pathloss. In the experiments performed, this achieved up to 7× TX power saving (translating to t prolonged battery lifetime), and twice the requested power satisfied. Meanwhile, the simplified driving circuits of the power transmitter 201 high voltage driver relative to employing an arrangement with a level-shifter, biasing circuit and buffers was shown to save 23% of the total TX system power, while supporting up to 20× power delivery.

The described embodiment should not be construed as limitative.

Although tuning of the frequency is described above as being performed prior to adjustment of the duty cycle, it is envisaged that both may be performed simultaneously.

While only one electrode 209 coupling the power output module 205 to the user's body 105 and one electrode coupling the data receiver 207 to the user's body 105 are described above, it is envisaged that there may be two or more electrodes coupling the power output module 205 to the user's body 105 and/or two or more electrodes coupling the data receiver 207 to the user's body 105. For example, it is envisaged that multiple electrodes may be connected to corresponding HV driver outputs (from a plurality of HV drivers) to boost the body-coupled power or to form a specific transmission pattern. Likewise, multiple power receiver electrodes may be used with multiple rectifiers for improved power input. Multiple data receiver/transmitter electrodes may also or alternatively be used to adapt the input/output data strength, or to tailor towards different frequency bands.

While duty cycle adaptation is described above as being periodic, it is envisaged that instead it may be event-triggered. Examples of triggering events may include an abnormal data occurrence (from the sensor front-end) indicating an abnormal body signal (e.g. abnormal ECG, seizure, low/high body temperature). Such events may trigger more chip task or request more chip performance, in response to which, the duty cycle adaptation could be triggered to avoid power shortage.

While a dual-source power converter 317 is described above, it is envisaged that a power converter that does not concurrently convert the harvest energy and the transmitted power may be employed. For example, two separate converters may be employed, one for transmitted power and one for harvested energy.

While the three power receivers are described as being configured to harvest ambient energy coupled to the body as well as receiving transmitted power, it is envisaged that one or more of the power receivers may only receive transmitted power.

While three receivers are described above as part of the energy transfer apparatus 100, it is envisaged that greater or fewer receivers may be employed, for example, only a single power receiver may be employed.

While high voltage generation is described above without the use of a level-shifter, biasing circuit or buffers it is envisaged that in a variation one or all of these may be employed.

Although the dual-source power converter 317 is described as being employed in combination with the adjustment of the frequency and duty cycle of the transmitted power, it is envisaged that a dual-source power converter may be employed with a power source with a fixed frequency and/or duty cycle.

While a regulated voltage of 1.1V is described above, it is envisaged that other regulated voltages may be employed.

Having now fully described the invention, it should be apparent to one of ordinary skill in the art that many modifications can be made hereto without departing from the scope as claimed.

1. A power transmitter, comprising:
(i) a signal generator operable to generate an electrical signal comprising an adjustable frequency and duty cycle;
(ii) one or more first transmitter electrodes arranged to be electrically coupled to a living being body for transmission of the electrical signal via the living being body to at least one power receiver;
(iii) one or more second transmitter electrodes arranged to be electrically coupled to the living being body, and operable to receive an indication of power received by the at least one power receiver via the living being body; and
(iv) a controller configured to control the signal generator to adjust the frequency and duty cycle of the electrical signal in response to the indication of power received.

2. A power transmitter according to claim 1,
wherein the electrical signal comprises a plurality of components,
the one or more first electrodes are arranged to be electrically coupled to the living being body for transmission of each of the plurality of components of the electrical signal via the living being body to a corresponding one of a plurality of power receivers,
the one or more second electrodes are operable to receive an indication of power received by each of the plurality of power receivers via the living being body, and
the controller is configured to control the signal generator to adjust a component frequency and a component duty cycle of each of the plurality of components of the electrical signal in response to the indication of power received by the corresponding power receiver.

3. A power transmitter according to claim 1, wherein the signal generator further comprises a power generating circuit including:
a voltage multiplier circuit arranged to receive an input supply voltage, the voltage multiplier circuit comprising a plurality of cascaded voltage multipliers arranged to generate corresponding multiplier output signals; and
a driving circuit configured to generate the electrical signal with a specific output voltage swing in direct response to the corresponding multiplier output signals, the specific output voltage swing being greater than the input supply voltage.

4. A power transmitter according to claim 3, wherein the corresponding multiplier output signals include a driver supply voltage, at least one intermediate biasing signal, and a plurality of clock signals for the driving circuit.

5. An energy transfer apparatus comprising:
(i) a power transmitter according to claim 1; and
(ii) the at least one power receiver, comprising:
(a) one or more power receiver electrodes arranged to be electrically coupled to the living being body, the one or more power receiver electrodes operable to receive the transmitted electrical signal via the body;
(b) at least one rectifier for rectifying the transmitted electrical signal into at least one rectified electrical signal;
(c) a DC-DC converter operable to convert the at least one rectified electrical signal to generate a recovered power signal, wherein the DC-DC converter is operable to determine the power received by the at least one power receiver; and
(d) one or more data transmitter electrodes arranged to be electrically coupled to the living being body for transmission of the indication of the power received by the at least one power receiver to the power transmitter.

6. An energy transfer apparatus according to claim 5, wherein the one or more power receiver electrodes are further operable to receive a body-coupled ambient energy electrical signal via the body.

7. An energy transfer apparatus according to claim 6, wherein the at least one power receiver further includes:
a first rectifier for rectifying the transmitted electrical signal into a first rectified electrical signal;
a second rectifier for rectifying the body-coupled ambient energy electrical signal into a second rectified electrical signal, and wherein
the DC-DC converter is operable to convert the first rectified electrical signal and the second electrical signal concurrently to generate the recovered power signal.

8. An energy transfer apparatus according to claim 5, wherein the at least one power receiver further comprises a controller, wherein the controller is arranged to adjust an inductor charging time of the DC-DC converter to enable an input power of the DC-DC converter to reach a maximum value.

9. An energy transfer apparatus according to claim 5, wherein the DC-DC converter includes a power evaluation circuit and is operable between a load regulation mode for delivering regulated power to a load and a power evaluation mode for determining the power received by the power receiver.

10. An energy transfer apparatus according to claim 9, wherein the DC-DC converter is operable between the load regulation mode and the power evaluation mode responsive to a voltage level of the recovered power signal.

11. An energy transfer apparatus according of claim 5, wherein the power received by the at least one power receiver is determined as a function of an inductor discharge time of the DC-DC converter.

12. An energy transfer apparatus according claim 5, wherein the power received by the at least one power receiver is determined as an average received power.

13-15. (canceled)

16. A method of transmitting electrical power via one or more first transmitter electrodes coupled to a body of a living being, the method comprising:
generating, by a signal generator having an adjustable frequency and duty cycle, an electrical signal;
receiving an indication of power received by at least one power receiver via one or more second transmitter electrodes coupled to the living being body; and
adjusting the frequency and duty cycle of the electrical signal in response to the indication of power received.

17. A method of transmitting electrical power according to claim 16, further comprising
generating the electrical signal with a plurality of components, each component operable to transmit electrical power to a different power receiver of a plurality of power receivers;
receiving an indication of power received by each of the plurality of power receivers via the one or more second transmitter electrodes;
adjusting a component frequency and a component duty cycle of each of the plurality of components of the electrical signal in response to the indication of power received by the corresponding power receiver.

18. A method of transmitting electrical power according to claim 17,
further comprising time-division multiplexing the plurality of components to generate the electrical signal.

19. A method of transmitting electrical power according to claim 16, further comprising:
receiving an input supply voltage at a voltage multiplier circuit, the voltage multiplier circuit comprising a plurality of cascaded voltage multipliers; generating, by the plurality of cascaded voltage multipliers, corresponding multiplier output signals; and
generating the electrical signal with a specific output voltage swing in direct response to the corresponding multiplier output signals, the specific output voltage swing being greater than the input supply voltage.

20. A method of transmitting electrical power according to claim 19, wherein the corresponding multiplier output signals include a driver supply voltage, at least one intermediate biasing signal, and a plurality of clock signals for a driving circuit.

21. A method of transferring energy via a body of a living being, the method comprising:
transmitting, from a power transmitter, electrical power via one or more first transmitter electrodes coupled to the body of a living being according to a method of claim 16;
receiving, at the power receiver, the transmitted electrical signal via one or more power receiver electrodes coupled to the living being body;
rectifying the electrical signal into at least one rectified electrical signal;
converting the at least one rectified electrical signal to generate a recovered power signal;
determining the power received by the power receiver; and
transmitting the indication of the power received to the power transmitter via one or more data transmitter electrodes coupled to the living being body.

22. A method of transferring energy via a body of a living being according to claim 21, comprising
converting, at a DC-DC converter, the at least one rectified electrical signal to generate the recovered power signal, and
adjusting an inductor charging time of the DC-DC converter to enable an input power of the DC-DC converter to reach a maximum value.

23. A method of transferring energy via a body of a living being according to claim 22, further comprising determining the power received by the power receiver at the DC-DC converter or determining the power received by the power receiver at the DC-DC converter as a function of an inductor discharge time of the DC-DC converter.

24-31. (canceled)
